(19) United States
(12) Patent Application Publication
(10) Pub. No.: US 2009/0069267 A1
(43) Pub. Date: Mar. 12, 2009

(54) CENTRAL ADMINISTRATION OF STABLE FORMULATIONS OF THERAPEUTIC AGENTS FOR CNS CONDITIONS

(76) Inventors: Daniel J. Abrams, Denver, CO (US); Raymond Bunch, Phoenix, AZ (US); Tom Anchordoquy, Lakewood, CO (US); Karen E. Stevens, Westminster, CO (US)

Correspondence Address: MORRISON & FOERSTER LLP, 755 PAGE MILL RD, PALO ALTO, CA, 94304-1018, US

(21) Appl. No.: 12/180,373

(22) Filed: Jul. 25, 2008

Related U.S. Application Data
(63) Continuation-in-part of application No. 12/161,149.
(60) Provisional application No. 60/966,554, filed on Jul. 25, 2007.
(60) Provisional application No. 60/759,821, filed on Jan. 17, 2006.
(60) Provisional application No. 60/825,547, filed on Sep. 13, 2006.

(51) Int. Cl. A61K 31/7076; A61K 31/551; A61K 31/519; A61K 31/137; A61K 31/55; A61P 25/28

(52) U.S. Cl. 514/46; 514/220; 514/259.41; 514/651; 514/217; 514/211.13

(57) ABSTRACT

The present invention concerns compositions, methods and/or apparatus of central administration of various CNS-active agents. In particular embodiments, intrathecal administration is advantageous for decreasing the systemic concentrations of CNS agent, thereby decreasing side effect toxicity, while allowing more effective delivery of the agent to the site of action, simultaneously decreasing the dosage delivered to the subject. In particular embodiments, ICV delivery may be of use for patients who have previously proven to be refractory to systemic administration of CNS agents, in some cases due to systemic side effects, or for those patients whose symptoms are of sufficient severity to warrant more aggressive therapeutic intervention. ICV administration allows not only lower systemic concentration but also higher therapeutically effective concentration within the CNS.

### CROSS REFERENCE TO RELATED APPLICATIONS

This application claims the benefit of the filing date of U.S. provisional application No. 60/966,554, filed on Jul. 25, 2007 (which was originally filed as U.S. utility application Ser. No. 11/881,401, on Jul. 25, 2007 and then converted to a provisional application on Oct. 22, 2007). This application is also a continuation-in-part application of U.S. utility application Ser. No. 12/161,149, filed Jul. 16, 2008, which is a National Phase filing under 35 U.S.C. § 371 of International Application No. PCT/US2007/001201, filed on Jan. 17, 2007, which claims the benefit of the filing date of U.S. provisional application No. 60/759,821, filed on Jan. 17, 2006, and U.S. provisional application No. 60/825,547, filed on Sep. 13, 2006. The entire contents of each of the aforementioned applications are specifically hereby incorporated by reference for all purposes.

### FIELD OF THE INVENTION

The present invention relates to pharmaceutical compositions and methods of use, and more particularly to pharmaceutical compositions specifically formulated for use in central administration.

### BACKGROUND OF THE INVENTION

Lumbar continuous intrathecal treatment has been used routinely and frequently for more than 10 years. Greater than 50,000 child and adult patients in the US have had this mode of therapy for pain, spasticity, and to a very limited extent, for neoplasia, since the 1980s (see world wide web at medtronic.com/neuro/paintherapies/pain_treatment ladder/drug_infusion/dmg_dmg_deliv.html). Integrated catheter and computerized pump delivery systems are commercially available through several vendors, and several new microinjection systems are in development. The primary vendor is Medtronic, with the Synchromed-II system in routine use.

A recent report of supracerebellar intrathecal administration for medically refractory pain patients reported that most of the patients so treated responded to the intrathecal medications though they did not respond to peripheral medications. The available computerized pump and catheter devices used for pain and spasticity are surgically implanted through a lumbar puncture and placed subcutaneously in the abdomen. The devices are implanted chronically and are expected to remain in place for many years because of the chronicity of pain and spasticity. The computerized delivery offers additional patient benefits because it only needs to be filled every 3 months, and a computerized pump allows complex dosing options.

On an individual case basis, single- or multiple-dose intrathecal cranial injections have been used for years to treat CNS infections by neurosurgeons injecting antifungals and antibacterials with Ommaya reservoirs and intraventricular catheters in a saline or equivalent carrier at neutral pH.

Current medications used for long term spinal intrathecal drug delivery include fentanyl, sufentanil, meperidine, morphine, baclofen, ziconitide, clonidine and bupivacaine, with several, including gabapentin and BDNF, under investigation. (Anderson et al. 1999. Paice et al., 1996, Levy R 1997). All the medications are water soluble, are presented at a neutral pH and are mixed in isotonic salts without buffers or solubilizing agents. There are no drugs specifically approved for ICV use although chemotherapeutics (including cytarabine, methotrexate) and antimicrobials (including amphotericin B) have been used intermittently (Pickering et al. 1978). Current parenteral formulations do not consider the special requirements for safely solubilizing and stablilizing hydrophobic compounds for delivery into the ventricle.

Schizophrenia is a significantly disabling illness which is frequently ineffectively treated. One of the primary reasons for ineffective treatment of schizophrenia is the significant drawbacks of state-of-the-art antipsychotics as currently used. Ineffective treatment results from medication side effects, failure to achieve therapeutic doses, and problems with patient compliance. Prospective studies, with up to twenty years of follow-up, have demonstrated that 50-70% of schizophrenia patients have a persistent and chronic course of therapeutic treatment with only 20-30% of these patients able to lead somewhat normal lives (Fleischhaker et al. 2005, Walker et al. 2004). Failure to improve contributes to suicide attempts of up to 50% of patients. Between 5.6% and 13% of patients with schizophrenia will die from suicide (Marts 1992, Caldwell, et al. 1992, Levin 2005).

The overall U.S. 2002 cost of schizophrenia was estimated to be $62.7 billion, with $22.7 billion excess direct health care cost ($7.0 billion outpatient, $5.0 billion drugs, $2.8 billion inpatient, $8.0 billion long-term care) (Wu et al. 2005). Oral and intramuscular treatments have limited ability to overcome the efficacy problems of current pharmacologic therapies because of significant systemic side effects among other limitations.

Despite representing just 1% of the population (app. 2.2 million Americans), persons with schizophrenia represent 10% of the totally and permanently disabled population (reviewed in Rupp and Keith 1993, Narrow 1998). Per-capita Medicare and Medicaid expenditures for schizophrenia are greater than for non-psychiatric medical disorders across the adult lifespan (Bartels et al. 2003). According to the National Institute of Mental Health-sponsored Epidemiologic Catchment Area (ECA) study, lifetime prevalence of schizophrenia is 1.3% of the population. Schizophrenia is predominantly a degenerative condition marked by diminished independence, diminished neurological function and profound suffering. It is generally estimated that today only approximately 10% to 15% of people who have schizophrenia are able to also maintain full-time employment of any type (Wu et al. 2005). The predominant deficits in schizophrenia in executive function, secondary verbal memory, immediate verbal memory and vigilance lead to difficulties with socialization, problem solving and daily activities (Compi et al. 1988, Harding et al. 1987, Klonoff et al. 1970).

State of the art antipsychotic medications are administered in oral and long acting intramuscular (IM) forms and include newer atypical antipsychotics and older typical antipsychotics. Clozapine is one of the most effective of the oral atypical antipsychotic medications, with superior improvement in positive and negative symptoms in the treatment of refractory schizophrenia, and in reducing the risk of patient suicide (Reid et al. 1998, Volvavka et al. 2002, Azorin et al. 2001, Buchanan et al. 1998, Iqbal et al. 2003)). Unfortunately, clozapine has a 1% incidence of agranulocytosis and a 3% incidence of neutropenia (Atkin et al. 1996, Alvir et al. 1993), a potentially lethal effect of systemic administration which limits clozapine's use. Because of clozapine's superior efficacy, reduction of clozapine's toxicity would make it a highly effective medication for widespread use in medically refractory schizophrenic patients.

Clozapine is administered twice a day, has extensive first pass metabolism and its dose is slowly escalated over time to achieve efficacy. Clozapine's efficacy in treatment of refractory schizophrenia has been thoroughly studied and it is a superior medication when compared with other typical and atypical antipsychotics. Clozapine has been found to be superior in treatment of disabling negative symptoms that include disorganization, cognitive dulling and socialization (Volvavka et al. 2002, Azorin et al. 2001, Buchanan et al. 1998). Clozapine is superior in treatment of refractory schizophrenia. Eighty percent of patients switched from clozapine to other atypical antipsychotics will relapse into psychosis (Buchanan et al. 1998). Clozapine prevents aggression and suicide in schizophrenic patients better than other medications (Reid et al. 1998, Volvavka et al. 2002, Azorin et al. 2001, Buchanan et al. 1998, Iqbal et al. 2003). Clozapine reduces relative risk of suicidal behavior by a mean relative risk reduction from 3 up to 15. Despite its efficacy, 17% of patients discontinue clozapine due to systemic side effects (Iqbal et al. 2003), including hematologic (agranulocytosis, eosinophilia, leukocytosis, thrombocytosis, and acute leukemia), cardiovascular effects (myocarditis, cardiomyopathy, deep vein thrombosis and orthostatic hypotension), metabolic effects (weight gain, diabetes) and gastrointestinal system complications (see reports of death secondary to constipation, toxic hepatitis, and pancreatitis—Iqbal et al. 2003). Despite aggressive monitoring techniques 464 patients have developed agranulocytosis prior to 1996 and 13 of those patients died (Iqbal et al. 2003).

Both typical and atypical antipsychotics of use for schizophrenia have multiple significant side effects which include movement disorders, hypotension (typicals) and diabetes (atypicals). Other significant problems include extremely poor compliance with oral medications for schizophrenic medications. Intramuscular formulations, (including Resperidone and Olanzapine for the atypicals, and haloperidol in the typicals), are limited by the inability to halt medication once it is injected, “constant dosing”, and still significant systemic side effect profile. Transdermal systems under development may improve compliance, eliminate the pain of an intramuscular injection, and potentially can be discontinued abruptly, but still have the limitations of constant dosing and significantly unaltered side effect profiles. Side effect profiles are the most profound issue in antipsychotic administration, as side effects can result in patient death (e.g., bone marrow failure with clozapine) and patient illness (e.g., liver toxicity and cardiac conduction deficits).

### BRIEF SUMMARY OF THE INVENTION

The present invention provides methods, formulations, apparatuses containing one or more compositions, and kits containing compositions, for central delivery of therapeutic agents for central nervous system conditions. These conditions include schizophrenia, multiple sclerosis and epilepsy. The discussion of schizophrenia, and therapeutic agents administered to treat schizophrenia, are exemplary and are not intended to limit the invention, which includes methods, compositions, apparatus, and kits for the treatment of other CNS conditions without limitation.

More particularly, the present invention relates to methods, compositions, apparatus and kits for central administration of stabilized therapeutic agents for treatment of central nervous system (CNS) conditions, including but not limited to Alzheimer's disease, dementia, anxiety, schizophrenia, pain, drug addiction, bipolar disorder, anxiety, major depressive disorder (MDD), depression, sleep disorders, encephalitis, multiple sclerosis, closed head injury, Parkinson disease, brain tumors and epilepsy.

In certain embodiments, the compositions for stabilized therapeutic agents may comprise any known CNS-active therapeutic agent. Compositions may be designed that are soluble and stabilized for long-term storage, for example in a fluid reservoir of an intrathecal delivery apparatus. These compositions may be provided in kits containing the composition in solution or in dry form in an appropriate receptacle. The kit may also contain a pharmaceutically acceptable excipient and instructions for use. The kit may also contain an appropriate delivery apparatus for delivering the composition to the individual to be treated, or an appropriate device for delivering the composition to a fluid reservoir of a pump system. An appropriate receptacle for the composition may be a fluid reservoir that can be used as part of a delivery apparatus.

In accordance with certain aspects of the invention, an intrathecal delivery apparatus or an intracerebroventricular delivery apparatus may comprise a pump, fluid reservoir, monitoring system, a programmable control system, a catheter (such as an intrathecal or intracerebroventricular catheter), a battery and/or other elements known in the art.

In yet other aspects of the invention, methods for central administration, e.g., intrathecal delivery, of CNS-active therapeutic agents are provided. Such methods may comprise centrally administering a stabilized composition to a subject in need thereof. In certain embodiments, the methods may comprise, obtaining a stabilized composition of a CNS-active agent, storing the stabilized composition in a delivery apparatus (for example an intrathecal or intracerebroventricular delivery apparatus), and delivering centrally (for example, intrathecally or intracerebroventricularly) measured amounts of the agent at predetermined time intervals. In certain embodiments, central delivery (for example, intrathecal delivery or intracerebroventricular delivery) may be particularly efficacious in patients who have been found to be refractory to standard systemic administration of CNS-active agents. In more particular embodiments, patients who have failed two or more standard systemic therapies or whose conditions are severe enough to warrant more aggressive treatment than standard systemic therapies may benefit from central delivery (for example, intrathecal delivery or intracerebroventricular delivery).

In one aspect, the invention features methods for treating a CNS-related condition or disorder in a subject (such as a human) in need thereof. In some embodiments, the method includes intracerebroventricularly administering to the subject a pharmaceutical composition comprising (i) a CNS therapeutic agent effective to treat the CNS-related condition or disorder and (ii) a solubility enhancing agent. In some embodiments, the solubility enhancing agent allows an effective amount of the CNS therapeutic agent to be intracerebroventricularly administered to the subject. In some embodiments, the CNS therapeutic agent maintains solubility (e.g., the CNS therapeutic agent does not precipitate) in the composition for at least two months at physiological temperature and pH. In some embodiments, the method includes administering an antioxidant to the subject.

In one aspect, the invention provides apparatus. In some embodiments, the apparatus includes (i) an intracerebroventricular delivery device, (ii) a central nervous system (CNS) therapeutic agent, and (iii) a solubility enhancing agent. In some embodiments, the solubility enhancing agent allows an effective amount of the CNS therapeutic agent to be intracerebroventricularly administered to a subject (such as a human). In some embodiments, the CNS therapeutic agent maintains solubility in the presence of the solubility enhancing agent for at least two months at physiological temperature and pH. In some embodiments, the CNS therapeutic agent and/or the solubility enhancing agent are within the intracerebroventricular delivery device. In some embodiments, the CNS therapeutic agent and/or the solubility enhancing agent are not located within the intracerebroventricular delivery device. For example, the CNS therapeutic agent and/or the solubility enhancing agent may be located within a container (such as a vial) that is separate from the intracerebroventricular delivery device. In some embodiments, the apparatus includes an antioxidant. In some embodiments, the apparatus also includes a penetration enhancing excipient, such as an excipient that avoids, binds, or masks a glycoprotein pump. In some embodiments, the apparatus includes a syringe for transferring the CNS therapeutic agent and/or solubility enhancing agent from a container into the intracerebroventricular delivery device. In some embodiments, the apparatus includes a syringe that is preloaded with the CNS therapeutic agent and/or solubility enhancing agent. In some embodiments, the apparatus includes a measuring device (such as a ruler) for measuring tubing (such as a catheter) to ensure that an appropriate amount of tubing is used so that the CNS therapeutic agent is administered in the proper location. In some embodiments, the apparatus includes a CNS catheter that is capable of being used with an imaging system (such as an imaging system for determining whether the tip of the catheter is placed in the desired location). In some embodiments, the apparatus includes a sheath, such as a sheath that facilitates endoscopic confirmation of the location of the catheter tip. In some embodiments, the apparatus includes a sterilization agent, such as an antibiotic. In some embodiments, the antibiotic is used to irrigate and/or bathe the intracerebroventricular delivery device and/or catheter. In some embodiments, the apparatus includes a unit dosage form of the CNS therapeutic agent and/or solubility enhancing agent. These unit dosage forms can be stored in a suitable packaging in single or multiple unit dosages and may also be further sterilized and sealed. In some embodiments, the apparatus also includes instructions for using the apparatus to treat a CNS-related condition or disorder in a subject (such as a human). In some embodiments, the apparatus includes written instructions on a label or package insert (e.g., a paper sheet included in the apparatus) or machine-readable instructions (e.g., instructions carried on a magnetic or optical storage disk). In some embodiments, the instructions relating to the use of a CNS therapeutic agent includes information as to dosage, dosing schedule, and route of administration for the intended treatment. In some embodiments, the instructions include a description of selecting a subject suitable for treatment. The containers may be unit doses, bulk packages (e.g., multi-dose packages) or sub-unit doses. For example, an apparatus may also be provided that contains sufficient dosages of a CNS therapeutic agent disclosed herein to provide effective treatment for a subject for an extended period, such as about any of a week, 2 weeks, 3 weeks, 4 weeks, 6 weeks, 8 weeks, 3 months, 4 months, 5 months, 6 months, 7 months, 8 months, 9 months, or more. The apparatus may also include multiple unit doses of a CNS therapeutic agent and instructions for use and may be packaged in quantities sufficient for storage and use in pharmacies, for example, hospital pharmacies and compounding pharmacies.

In one aspect, the invention features kits. In some embodiments, the kit includes (i) an intracerebroventricular delivery device, (ii) an amount of a central nervous system (CNS) therapeutic agent suitable for intracerebroventricular administration to a subject (such as a human) in need thereof, and (iii) a solubility enhancing agent. In some embodiments, the kit includes instructions for using the kit to treat a CNS-related condition or disorder in the subject. In some embodiments, the solubility enhancing agent allows an effective amount of the CNS therapeutic agent to be intracerebroventricularly administered to the subject. In some embodiments, the CNS therapeutic agent maintains solubility in the presence of the solubility enhancing agent for at least two months at physiological temperature and pH. In some embodiments, the CNS therapeutic agent and the solubility enhancing agent are located within the same container (such as a vial). In some embodiments, the CNS therapeutic agent and the solubility enhancing agent are located within separate containers. In some embodiments, the kit includes an antioxidant. In some embodiments, the kit also includes a penetration enhancing excipient, such as an excipient that avoids, binds, or masks a glycoprotein pump. In some embodiments, the kit includes a syringe for transferring the CNS therapeutic agent and/or solubility enhancing agent from a container into the intracerebroventricular delivery device. In some embodiments, the kit includes a syringe that is preloaded with the CNS therapeutic agent and/or solubility enhancing agent. In some embodiments, the kit includes a measuring device (such as a ruler) for measuring tubing (such as a catheter) to ensure that an appropriate amount of tubing is used so that the CNS therapeutic agent is administered in the proper location. In some embodiments, the kit includes a CNS catheter that is capable of being used with an imaging system (such as an imaging system for determining whether the tip of the catheter is placed in the desired location). In some embodiments, the kit includes a sheath, such as a sheath that facilitates endoscopic confirmation of the location of the catheter tip. In some embodiments, the kit includes a sterilization agent, such as an antibiotic. In some embodiments, the antibiotic is used to irrigate and/or bathe the intracerebroventricular delivery device and/or catheter. In some embodiments, the kit includes a unit dosage form of the CNS therapeutic agent and/or solubility enhancing agent. These unit dosage forms can be stored in a suitable packaging in single or multiple unit dosages and may also be further sterilized and sealed. In some embodiments, the kit includes written instructions on a label or package insert (e.g., a paper sheet included in the kit) or machine-readable instructions (e.g., instructions carried on a magnetic or optical storage disk). In some embodiments, the instructions relating to the use of a CNS therapeutic agent includes information as to dosage, dosing schedule, and route of administration for the intended treatment. In some embodiments, the instructions include a description of selecting a subject suitable for treatment. The containers may be unit doses, bulk packages (e.g., multi-dose packages) or sub-unit doses. For example, kits may also be provided that contain sufficient dosages of a CNS therapeutic agent disclosed herein to provide effective treatment for a subject for an extended period, such as about any of a week, 2 weeks, 3 weeks, 4 weeks, 6 weeks, 8 weeks, 3 months, 4 months, 5 months, 6 months, 7 months, 8 months, 9 months, or more. The kits may also include multiple unit doses of a CNS therapeutic agent and instructions for use and may be packaged in quantities sufficient for storage and use in pharmacies, for example, hospital pharmacies and compounding pharmacies.

In one aspect, the invention features the use of a CNS therapeutic agent in the manufacture of a medicament for treatment of a CNS-related condition or disorder in a subject (such as a human) in need thereof. In one aspect, the invention features the use of a CNS therapeutic agent for treating a CNS-related condition or disorder in a subject (such as a human) in need thereof. In some embodiments, the medicament further comprises a solubility enhancing agent that allows an effective amount of the CNS therapeutic agent to be intracerebroventricularly administered to the subject. In some embodiments, the solubility enhancing agent maintains the CNS therapeutic agent in solution for at least two months at physiological temperature and pH to thereby accommodate chronic intracerebroventricular administration to the subject. In some embodiments, the medicament is formulated so as to accommodate chronic intracerebroventricular administration over at least two months via an implantable delivery device.

In some embodiments of any of the compositions, methods, apparatus, kits, and uses of the invention, the CNS-related condition or disorder is selected from the group consisting of epilepsy, schizophrenia, closed head injury spectrum, Alzheimer's disease spectrum, sleep disorders spectrum, depression, anxiety spectrum, bipolar disorder, and multiple sclerosis. In some embodiments, the subject is selected from the population of individuals who are refractory to treatment via systemic administration of the CNS therapeutic agent. In some embodiments, the refractory subject shows an alleviation of one or more symptoms when treated by intracerebroventricular administration of the pharmaceutical composition.

In some embodiments of any of the compositions, methods, apparatus, kits, and uses of the invention, the CNS therapeutic agent is active in the treatment of epilepsy. In some embodiments, the CNS therapeutic agent is an anti-epilepsy agent that acts on the GABA system, a sodium channel, and/or a calcium channel. In some embodiments, the CNS therapeutic agent is selected from the group consisting of felbamate, lamictal, bumex, tegretol, valproate, adenosine, pharmaceutically acceptable salts, esters, and acids thereof, and combinations thereof.

In some embodiments of any of the compositions, methods, apparatus, kits, and uses of the invention, the CNS therapeutic agent is active in the treatment of schizophrenia. In some embodiments, the CNS therapeutic agent is an anti-schizophrenic agent that acts as a nicotinic direct or indirect agonist, or a dopamine antagonist. In some embodiments, the CNS therapeutic agent is selected from the group consisting of clozapine, ondansetron, olanzapine, risperidone, pharmaceutically acceptable salts, esters, and acids thereof, and combinations thereof.

In some embodiments of any of the compositions, methods, apparatus, kits, and uses of the invention, the CNS therapeutic agent is active in the treatment of depression and/or anxiety. In some embodiments, the CNS therapeutic agent is an anti-depression and/or anti-anxiety agent that affects adrenergic and serotonergic activity. In some embodiments, the CNS therapeutic agent is selected from the group consisting of phenelzine, fluoxetine, tranylcypromine, amitryptyline, clomipramine, isocarboxazid, pharmaceutically acceptable salts, esters, and acids thereof, and combinations thereof.

In some embodiments of any of the compositions, methods, apparatus, kits, and uses of the invention, the subject is administered a dosage of the CNS therapeutic agent significantly reduced, as compared to the dosage required when administered systemically. In some embodiments, the CNS therapeutic agent is present at a concentration greater than corresponding concentrations suitable for systemic administration. In some embodiments, the dosage of CNS therapeutic agent is at an intracerebroventricular administration to systemic administration ratio of about 1:250 to about 1:600.

In some embodiments of any of the compositions, methods, apparatus, kits, and uses of the invention, the solubility enhancing agent is selected from the group consisting of cyclodextrin, octylglucoside, Tween 20, polyethylene glycol, sucrose ester, pluronic F-68, and combinations thereof. In some embodiments, the cyclodextrin is β-hydroxypropyl-cyclodextrin. In some embodiments, the CNS therapeutic agent is a hydrophobic compound.

In some embodiments of any of the compositions, methods, apparatus, kits, and uses of the invention, the CNS therapeutic agent to solubility enhancing agent molar ratio is between about 1:1 and about 1:10. In some embodiments, the CNS therapeutic agent maintains CNS therapeutic agent stability in cerebral spinal fluid upon intracerebroventricular administration to the subject. In some embodiments, the CNS therapeutic agent maintains solubility in cerebral spinal fluid upon intracerebroventricular administration to a subject. In some embodiments, the CNS therapeutic agent does not precipitate and is non-toxic while it remains in the in the cerebral spinal fluid. In some embodiments, the delivery device is an implantable pump. In some embodiments, the pharmaceutical composition is chronically administered over at least two months via an implantable delivery device.

In some embodiments of any of the compositions, methods, apparatus, kits, and uses of the invention, the subject is a human. In some embodiments, the intracerebroventricular administered dose to the human is about 30-fold lower to about 30-fold higher than the effective ICV dose in rats. In some embodiments, the intracerebroventricular administered dose to the human is about 20-fold lower to about 30-fold higher than the effective ICV dose in rats. In some embodiments, the intracerebroventricular administered dose to the human is about 10-fold lower to about 30-fold higher than the effective ICV dose in rats. In some embodiments, the intracerebroventricular administered dose to the human is about 10-fold lower to about 20-fold higher than the effective ICV dose in rats. In some embodiments, the intracerebroventricular administered dose to the human is about 10-fold lower to about 10-fold higher than the effective ICV dose in rats. In some embodiments, the CNS therapeutic agent is felbamate and the effective ICV dose in rats is about 0.08242 mg/kg. In some embodiments, the CNS therapeutic agent is clozapine and the effective ICV dose in rats is about 0.040 mg/kg.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 illustrates the solubility of clozapine at physiological pH in the presence of different solubility enhancing agents.

FIG. 2 illustrates the solubilization of clozapine at different cyclodextrin-to-clozapine molar ratios.

FIGS. 3A-3B illustrate toxicity data of clozapine in cyclodextrin.

FIGS. 4A-4B illustrate the effects of ICV administration of 0.5 μg of clozapine.

FIGS. 5A-5B illustrate the effects of ICV administration of 1 μg clozapine.

FIGS. 6A-6B illustrate the effects of ICV administration of 0.5 μg of ondansetron.

FIGS. 7A-7F, 8A-8B, 9A-9B, 10A-10B, 11A-11B and 12 illustrate the effects of ICV administration of various anti-depressants as well as cyclodextrin as a control.

### DETAILED DESCRIPTION OF THE INVENTION

In certain aspects, the present invention relates to compositions and methods including agents active in the treatment of central nervous system (CNS) conditions and disorders that are particularly suited for delivery via the cerebrospinal fluid (CSF). Further, in certain embodiments, the compositions and methods are surprisingly effective in the treatment of medically refractory patients.

In accordance with the embodiments of the present invention, it has been found desirable to formulate compositions of CNS-active therapeutic agents for central administration via, e.g., an intrathecal delivery device at relatively high concentrations so that small injection volumes will be sufficient to attain therapeutic drug levels within the CSF.

In other embodiments, it has been found that surprisingly small dosages may be used when the CNS-active therapeutic agents are administered centrally. More particularly, up to a 1:600 ICV to oral equivalency dose on a mg/kg basis, and a 1:125 ICV to IV equivalency are observed in accordance with certain embodiments of the invention (based on rodent model dosages and known mouse to human equivalency). These small dosages result in marked advantages in therapeutic outcome in terms of toxicity, side effects, dosing regimens, patient compliance, etc.

### A. Pharmaceutical Compositions:

One aspect is drawn to pharmaceutical compositions of CNS-active therapeutic agents suitable for central administration, particularly long term or chronic central administration, e.g., using implantable intrathecal pumps. The development of compositions for central administration, particularly long term or chronic central administration, has previously been a relatively unexplored field within the pharmaceutical sciences.

In certain aspects, the pharmaceutical compositions of the present invention allow for formulation of CNS-active therapeutic agents at higher dosage concentrations than typically used for systemic administration. As described in further detail below, the compositions of the present invention, in certain embodiments, provide for maximal solubility and stability under conditions of use during central administration, particularly chronic central administration. In this regard, it has been found in accordance with certain embodiments and aspects of the invention that the compositions, when administered via central administration routes, are suitable for use at higher dosage concentrations without increased risks of toxicity, as compared to systemic administration routes. In other embodiments and aspects, it has been found that significantly smaller amounts of the compositions of the present invention need to be centrally administered to achieve equipotent effect, as compared to systemic administration.

1. Exemplary CNS-Active Therapeutic Agents

Any suitable agent active in the treatment or prevention of a CNS condition, disease or disorder may be used in the context of the present invention. By way of non-limiting example, such agents include anti-epilepsy agent that acts on the GABA system, the Sodium Channel, and/or Calcium Channel that also have efficacy in bipolar disorder and closed head injury spectrum; anti-schizophrenic agent that acts as a nicotinic direct or indirect agonist, or a dopamine antagonist that also can have efficacy in closed head injury spectrum and Alzheimer disease spectrum; anti-depression and/or anti-anxiety agent that affects adrenergic and serotonergic activity that also can have efficacy in eating disorders and behavioral disorders, etc.

CNS-active therapeutic agents (herein also referred to as “active agents”) that may be formulated and centrally administered in accordance with the present invention include, but are not limited to, clozapine, felbamate (felbatol), adenosine (and analogues thereof, e.g., a1 and a2 agonists, a1 and a2 analogue agonists, etc.), phenyloin, lamictal, phenobarbital, ethosuximide, isocarboxazid, carbamezapine, valproic acid, progabide, clorazepate, Etobarb, oxezapam, alprazolam, bromazepam, chlordiazepoxide, clobazam, clonazepam, estazolam, flurazepam, halazepam, ketazolam, quazepam, prazepam, temazepam, triazolam, nitrazepam, carbatrol, hydroxyzine, oxcarbazepine, zarontin, lamotrigine, lithium, olanzapine, risperidone, seroquel, aripiprazole, ziprasidone, clozapine, haloperidol, chlorpromazine, loxitane, navane, mellaril, thorazine, moban, trilafon, prolixin, stelazine, Parnate, phenelzine, clomipramine, loxapine, thioridazine, thiothixine, prochlorperazine, trifluoperazine, fluphenazine, any other known antipsychotic, bromocriptine, L-Dopa, Zonisamide, methadone, buprinorphine, duramorph, clonidine, clonazapate, diazepam, temezapam, oxazepam, lorezapam, flurazepam, clonazepam, triazolam, chlordiazepoxide, alprazolam, Luvox, paroxetine, fluoxetine, amitryptiline, nortryptiline desipramine, amantadine, salicylic acid, ibuprofen, acetimonophen, sulfasalazine, dexamethasone, dihryoepiandrosterone, dexamethasone prednisilone, methylprestone, other known steroids, caffeine, cocaine, amphetamines, naloxone, methotrexate, 5-FU, methylprednisolone, cytosine arabinoside, other known cancer chemotherapeutic agents, cimetidine, famotidine, Nizatidine, ranitidine and any known antianxiety agents, pharmaceutically acceptable salts, esters, and acids thereof, and combinations thereof.

Additional active agents are shown in the table below, along with certain physical properties useful in selecting suitable solubility enhancing agents and/or stabilizing excipients. As generally understood by those skilled in the art, the listing of an active agent includes pharmaceutically acceptable salts, esters, and acids thereof.

Combinations of active agents, including secondary active agents effective to treat secondary indications, complications, or conditions are also envisioned. For instance, olanzapine is known to increase weight and adding a small amount of ICV stimulant (e.g., amphetamine) will offset the weight gain for patients. In addition, adding allopurinol, which is thought to be related to increased adenosine and antipsychotic activity, or adding adenosine directly with clozapine can decrease antipsychotic activity. However, the invention is not so limited, and any suitable synergistic or collaborative therapy known in the art may be used.

Table of active agents:

| Name | Chemical Name | Structure | Comments | Exemplary Indications |
|---|---|---|---|---|
| METHADONE | 1,1-Diphenyl-1-(2-dimethylaminopropyl)-2-butanone | 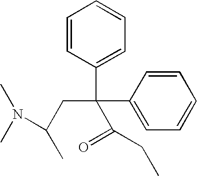 | Soluble in water;freely soluble inalcohol and inchloroform;practically insoluble inether and in glycerol | Addiction; PainDisorders; Anxiety |
| CLONIDINE | 2-(2,6-Dichloro phenylimino)imidazolidine | 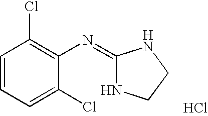 | | Addiction; PainDisorders; Anxiety |
| AMANTADINE | 1-adamantanaminehydrochloride | 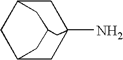 | Freely soluble inalcohol and in methylalcohol | Addiction; PainDisorders |
| VALPROIC ACID | 2-Propylpentanoic acid | 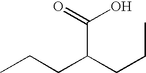 | | Addiction; PainDisorders; Anxiety;Depression;Schizophrenia;Bipolar Disorder;Epilepsy |
| BUPROPION | 1-(3-chlorophenyl)-2-[(1,1-dimethylethyl)amino]-1-propanone hydrochloride | 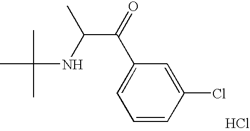 | freely soluble inwater and soluble inalcohol and inchloroform. | Addiction; Anxiety;Depression |
| CARBAMEZAPINE | 5-carbamoyl-5H-dibenz[b,f]azepine | 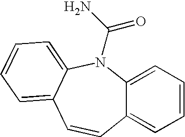 | | Addiction; PainDisorders;Depression;Schizophrenia;Bipolar Disorder;Epilepsy |
| ANTABUSE | bis(diethylthiocarbamoyl)disulfide | 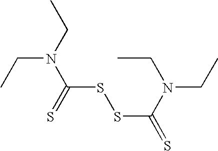 | Soluble in water <0.1g/100 mL at 22 C. | Addiction |
| CLOZAPINE | 8-chloro-11-(4-methyl-1-piperazinyl)-5H-dibenzo[b,e][1,4]diazepine | 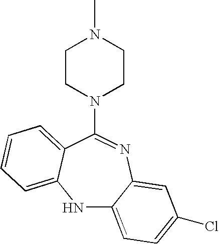 | | Addiction;Schizophrenia;Bipolar Disorder |
| LOREZAPAM | 7-chloro-5-(O-chlorophenyl)-1,3-dihydro-3-hydroxy-2H-1,4-benzo-diazepin-2-one | 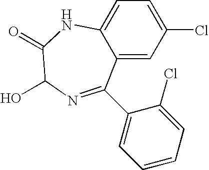 | Very soluble inorganic solvents. | Addiction; PainDisorders; Anxiety;Schizophrenia;Bipolar Disorder |
| FLURAZEPAM | 7-chloro-1-[2-(diethylamino)ethyl]-5-(o-fluorophenyl)-1,3-dihydro-2H-1,4-benzodiazepin-2-onedihydrochloride | 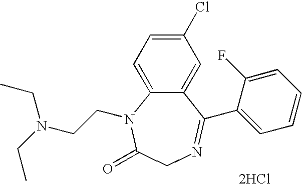 | Soluble in water, inalcohol, and in 0.1 Nhydrochloric acid. | Addiction; PainDisorders; Anxiety;Schizophrenia;Bipolar Disorder;Epilepsy |
| CLONAZEPAM | 5-(2-chlorophenyl)-1,3-dihydro-7-nitro-2H-1,4-benzodiazepin-2-one | 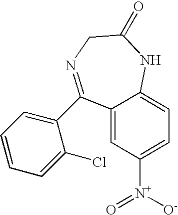 | | Addiction; PainDisorders; Anxiety;Schizophrenia;Bipolar Disorder;Epilepsy |
| TRIAZOLAM | 8-chloro-6-(o-chlorophenyl)-1-methyl-4H-s-triazolo-(4,3-alpha)(1,4)benzodiazepine | 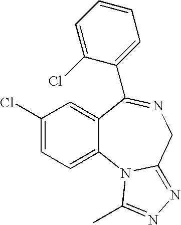 | practically insoluble inwater and soluble inalcohol and inacetone | Addiction; PainDisorders; Anxiety;Schizophrenia;Bipolar Disorder;Epilepsy |
| CHLORDIAZEPOXIDE | 7-chloro-2-(methylamino)-5-phenyl-3H-1,4-benzodiazepine 4-oxidehydrochloride | 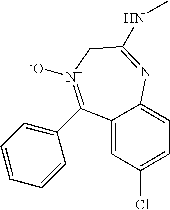 | | Addiction; PainDisorders; Anxiety;Schizophrenia;Bipolar Disorder |
| TEMAZEPAM | 7-chloro-1,3-dihydro-3-hydroxy-1-methyl-5-phenyl-2H-1,4-benzodiazepin-2-one | 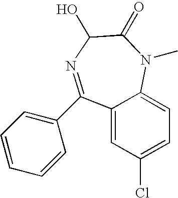 | Insoluble in water<0.01 g/100 mL at21 C. | Addiction; PainDisorders; Anxiety;Schizophrenia |
| OXEZAPAM | 7-chloro-1,3-dihydro-3-hydroxy-5-phenyl-2H-1,4-benzodiazepin-2-one | 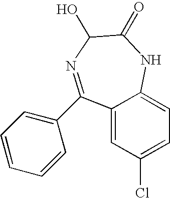 | | Addiction; PainDisorders; Anxiety;Schizophrenia;Bipolar Disorder |
| CLORAZEPATE | 7-Chloro-2,3-dihydro-2,2-dihydroxy-5-phenyl-1H-1,4-benzodiazepine-3-carboxylicacid | 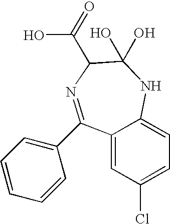 | | Addiction; PainDisorders; Anxiety;Schizophrenia;Bipolar Disorder;Epilepsy |
| DIAZEPAM | 7-chloro-1,3-dihydro-1-methyl-5-phenyl-2H-1,4-benzodiazepin-2-one | 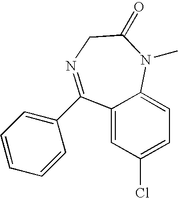 | C₁₀H₂₀N₂S₄ | Addiction; PainDisorders; Anxiety;Schizophrenia;Bipolar Disorder;Epilepsy |
| ALPRAZOLAM | 8-Chloro-1-methyl-6-phenyl-4H-s-triazolo(4,3-a)(1,4)benzodiazepine | 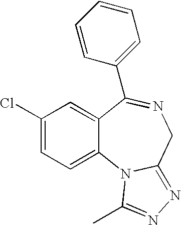 | very slightly solublein water | Addiction; PainDisorders; Anxiety;Bipolar Disorder |
| IBUPROFEN | 2-(p-isobutylphenyl)propionic acid | 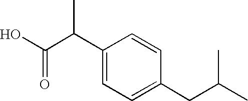 | | Addiction; PainDisorders |
| SULFASALAZINE | 5-([p-(2-pyridylsulfamoyl)phenyl]azo) salicylic acid | 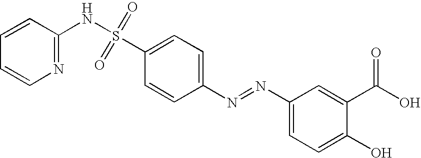 | C₁₈H₁₉ClN₄ MW326.83 | Addiction; PainDisorders |
| SALICYLIC ACID | 2-Hydroxybenzoic acid | 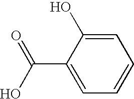 | almost insoluble inwater | Addiction; PainDisorders |
| ACETAMINOPHEN | 4-Acetamidophenol | 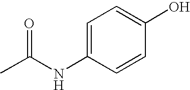 | | Addiction; PainDisorders |
| CAFERGOT | 12′-Hydroxy-2′-methyl-5′-(phenylmethyl)ergotaman-3′,6′,18-trione | 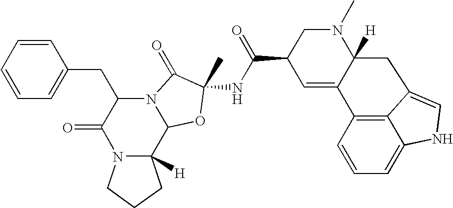 | Each ml of sterileAtivan injectioncontains either 2.0 or4.0 mg of lorazepam,0.18 ml polyethyleneglycol 400 inpropylene glycol with2.0% benzyl alcoholas preservative. | Addiction; PainDisorders |
| NALOXONE | (−)-17-Allyl-4,5α-epoxy-3,14-dihydroxymorphinan-6-one hydrochloride | 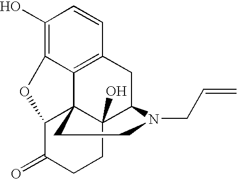 | freely soluble in USPalcohol and verysoluble in water | Addiction; PainDisorders |
| CITALOPRAM | 1-(3-Dimethylaminopropyl)-1-(4-fluorophenyl)-1,3-dihydroisobenzofuran-5-carbonitrile | 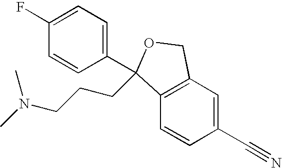 | Insoluble in water;slightly soluble inalcohol and in ether;sparingly soluble inacetone and inchloroform. | Anxiety; Depression |
| FLUVOXAMINE | (E)-5-Methoxy-4′-trifluoromethylvalerophenoneO-2-aminoethyloximemaleate | 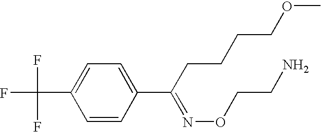 | | Anxiety; Depression |
| PAROXETINE | (−)-trans-4R-(4′-fluorophenyl)-3S-((3′,4′-methylenedioxyphenoxy)-methyl)piperidine | 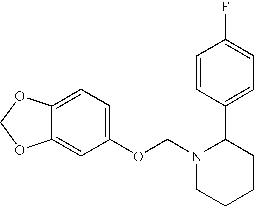 | soluble in alcohol andpoorly soluble inwater | AnxietyDepression |
| FLUOXETINE | (+/−)-N-Methyl-3-phenyl-3-(alpha,alpha,alpha-trifluoro-p-tolyloxy)propylaminehydrochloride | 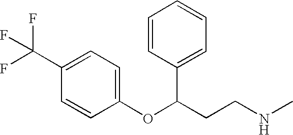 | soluble in water | AnxietyDepression |
| SERTRALINE | 1S-cis)-4-(3,4-dichlorophenyl)-1,2,3,4-tetrahydro-N-methyl-1-naphthalenamine | 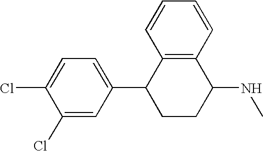 | (C₁₆H₁₄CIN₃O•HCL) | AnxietyDepression |
| DOXEPIN | (E)-3-(Dibenz[b,e]oxepin-11-ylidene)propyldimethylaminehydrochloride | 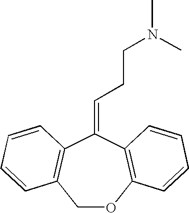 | | Anxiety |
| CLOMIPRAMINE | 3-(3-Chloro-10,11-dihydro-5H-dibenz[b,f]azepin-5-yl)propyldimethylaminehydrochloride | 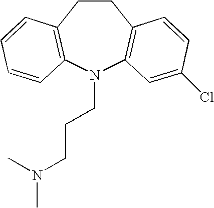 | It is unstable insolution and thepowder must beprotected from light | AnxietyDepression |
| NORTRIPTYLINE | Semicarbazidehydrochloride | 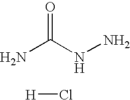 | very slightly solublein water andsparingly soluble inalcohol | AnxietyDepression |
| AMITRIPTILINE | 3-(10,11-Dihydro-5H-dibenzo[a,d]cyclohepten-5-ylidene)propyldimethylamine;10,11-Dihydro-N,N-dimethyl-5H-dibenzo[a,d]cycloheptene-delta(5,gamma)-propylamine | 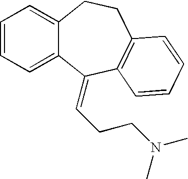 | | AnxietyDepression |
| MAPROTILINE | 3-(9,10-Dihydro-9,10-ethanoanthracen-9-yl)propyl(methyl)amine; N-Methyl-9,10-ethanoanthracene-9(10H)-propylamine | 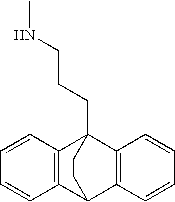 | C₁₆H₁₃ClN₂O₂ | AnxietyDepression |
| DESIPRAMINE | 3-(10,11-Dihydro-5H-dibenz[b,f]azepin-5-yl)propyl(methyl)aminehydrochloride | 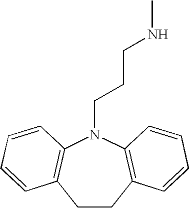 | MW 300.74 | AnxietyDepression |
| TRIMIPRAMINE | Dimethyl{3-(10,11-dihydro-5H-dibenz[b,f]azepin-5-yl-2-methyl)propyl}amine | 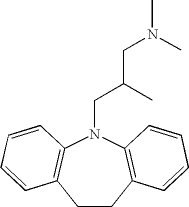 | (C₁₅H₁₁ClN₂O₂) | AnxietyDepression |
| IMIPRAMINE | 10,11-dihydro-N,N-dimethyl-5H-Dibenz[b,f]azepine-5-propanamine | 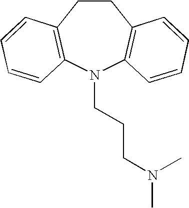 | insoluble in thecommon organicsolvents, but verysoluble in water. | AnxietyDepression |
| PROTRIPTYLINE | 3-(5H-Dibenzo[a,d]cyclohept-5-enyl)propyl(methyl)aminehydrochloride | 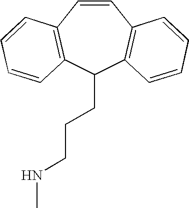 | | AnxietyDepression |
| ISOCARBOXAZID | 2′-Benzyl-5-methylisoxazole-3-carbohydrazide | 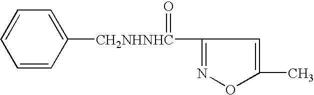 | Aqueous solutions areunstable, clear, lightyellow, and alkaline | Anxiety |
| PHENELZINE | 2-Phenylethylhydrazine | 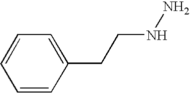 | | AnxietyDepression |
| TRANYLCYPROMINE | (+/−)-trans-2-Phenylcyclopropylaminesulphate | 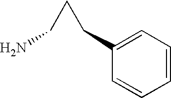 | C₁₆H₁₁ClK₂N₂O₄ | AnxietyDepression |
| TRAZODONE | 2-[3-(4-m-Chlorophenylpiperazin-1-yl)propyl]-1,2,4-triazolo[4,3-a]pyridin-3(2H)-one hydrochloride | 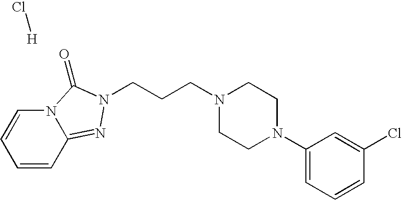 | insoluble in water<0.1 g/100 mL at 20C. | AnxietyDepression |
| BUSPIRONE | 8-[4-[4-(2-pyrimidinyl)-1-piperazinyl]butyl]-8-azaspiro[4,5]decane-7,9-dione | 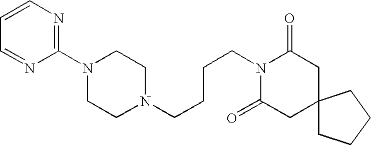 | | Anxiety |
| PROPRANOLOL | 1-(Isopropylamino)-3-(1-naphthyloxy)-2-propanolhydrochloride | 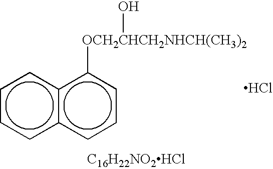 | soluble in methanolor ethanol but whichhas no appreciablesolubility in water atphysiological pH | Anxiety |
| ATENOLOL | 4-(2-Hydroxy-3-((1-methylethyl)amino)propoxy)-benzeneacetamide | 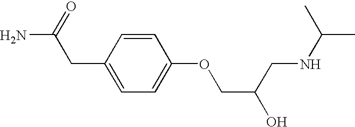 | | Anxiety |
| PRAZOSIN | 2-[4-(2-Furoyl)piperazin-1-yl]-6,7-dimethoxyquinazolin-4-ylamine hydrochloride | 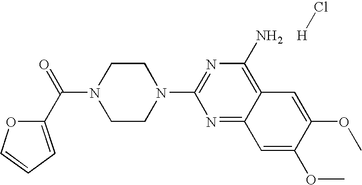 | very slightly solublein water (<1 mg/ml)and readily soluble inorganic solvents suchas ethanol andacetone | Anxiety |
| GUANFACINE | N-Amidino-2-(2,6-dichlorophenyl)acetamidehydrochloride | 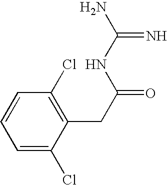 | | Anxiety |
| TRAMADOL | (+/−)-trans-2-Dimethylaminomethyl-1-(3-methoxyphenyl)cyclohexanolhydrochloride | 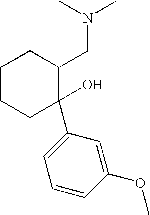 | white powder with amelting point of 74°-77°C. | Depression |
| NEFAZODONE | 2-(3-(4-(3-chlorophenyl)-1-piperazinyl)propyl)-5-ethyl-2,4-dihydro-4-(2-phenoxyethyl)-3H-1,2,4-triazol-3-one | 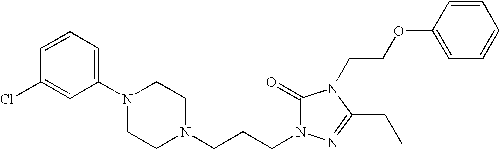 | Soluble in water <0.1g/100 mL at 25 C. | Depression |
| PERPHENAZINE | 4-(3-(2-Chlorophenothiazin-10-YL)propyl)-1-piperazineethanol | 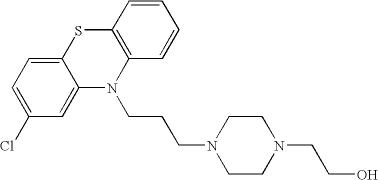 | | Depression |
| AMOXAPINE | 2-Chloro-11-(1-piperazinyl)dibenz(b,f)(1,4)oxazepine | 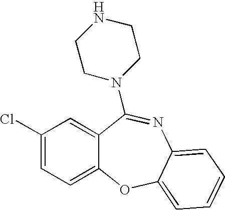 | Soluble 1 in 460 ofwater, 1 in 15 ofboiling water, 1 in 3of alcohol, 1 in 45 ofchloroform, 1 in 3 ofether, and 1 in 135 ofbenzene. | Depression |
| DOXEPIN | (E)-3-(Dibenz[b,e]oxepin-11-ylidene)propyldimethylaminehydrochloride | 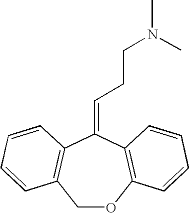 | | Depression |
| LITHIUM | molecular formula Li2CO3 | | Very slightly soluble | Depression |
| | | | in water 0.1-0.5 | Schizophrenia |
| | | | g/100 mL at 22 C. | Bipolar Disorder |
| RISERIDONE | 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one | 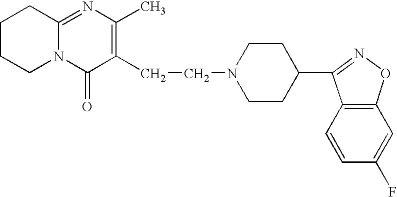 | | AnxietyDepressionSchizophreniaBipolar Disorder |
| CHLORPROMAZINE | 10-(3-dimethylaminopropyl)-2-chlorphenothiazine | 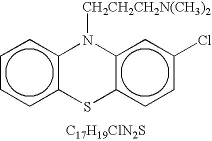 | | SchizophreniaBipolar Disorder |
| FLUPHENAZINE | 1-(2-Hydroxyethyl)-4-(3-(trifluoromethyl-10-phenothiazinyl)propyl)-piperazine | 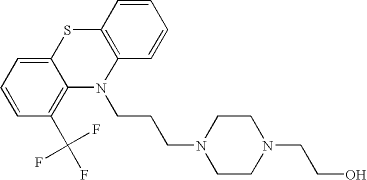 | soluble in water, indilute acids, and instrong alkali; slightlysoluble in alcohol;practically insoluble inether and inchloroform | SchizophreniaBipolar Disorder |
| HALOPERIDOL | 4-[4-(p-chlorophenyl)-4-hydroxypiperidino]-4′-fluorobutyrophenone | 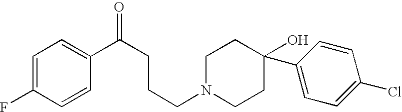 | sparingly soluble inwater and soluble inethanol | SchizophreniaBipolar Disorder |
| LOXAPINE | 2-chloro-11-(4-methyl-1-piperazinyl)dibenz[b,f][1,4]oxazepine | 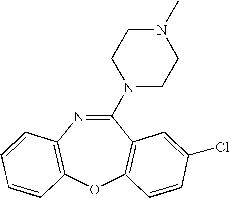 | Sparingly soluble inwater; freely solublein alcohol and inmethyl alcohol | SchizophreniaBipolar Disorder |
| THIORIDAZINE | 1-OH-Phenothiazine,10-[2-(1-methyl-2-piperidinyl)ethyl]-2-(methylthio)-monohydrochloride | 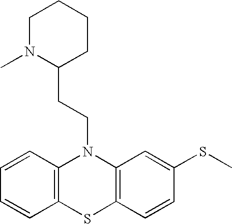 | | SchizophreniaBipolar Disorder |
| THIOTHIXINE | cis isomer of N,N-dimethyl-9-□3-(4-methyl-1-piperazinyl)-propylidene□thioxanthene-2-sulfonamide. | 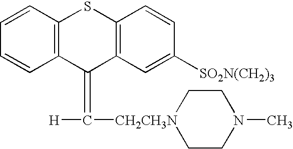 | Slightly soluble inwater; soluble inalcohol and in methylalcohol | SchizophreniaBipolar Disorder |
| PROCHLORPERAZINE | 2-chloro-10-[3-(4-methyl-1-piperazinyl)propyl]-10H -phenothiazine(Z)-2-butenedioate (1:2) | 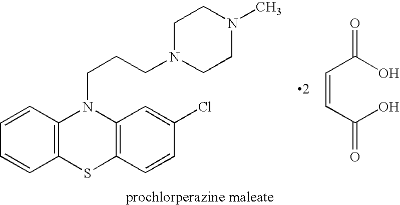 | | SchizophreniaBipolar Disorder |
| TRIFLUOPERAZINE | 10-[3-(4-Methylpiperazin-1-yl)propyl]-2-trifluoromethylphenothiazinedihydrochloride | 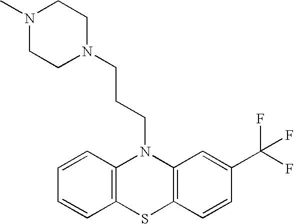 | Sparingly soluble inwater and indichloromethane;freely soluble inalcohol and in methylalcohol; practicallyinsoluble in ether | SchizophreniaBipolar Disorder |
| METHYLPRESTONE | | | | Schizophrenia |
| | | | | Bipolar Disorder |
| HYDROXYZINE | 2-[2-[4-(p-chlorobenzhydryl)-1-piperazinyl]ethoxy]ethanoldihydrochloride. | 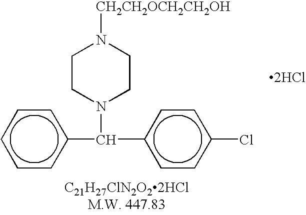 | slightly soluble inwater and isopropylalcohol, sparinglysoluble in ethanol | AnxietySchizophreniaBipolar Disorder |
| OXCARBAZEPINE | 10,11-Dihydro-10-oxo-5H-dibenz[b,f]azepine-5-carboxamide | 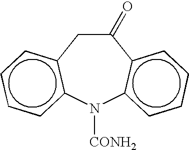 | Freely soluble inwater, in alcohol, andin dichloromethane | SchizophreniaBipolar Disorder |
| ETHOSUXIMIDE | 2-Ethyl-2-methylsuccinimide | 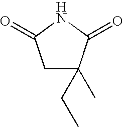 | | SchizophreniaBipolar Disorder |
| PHENYTOIN | 5,5-diphenyl-2,4-imidazolidinedione | 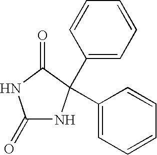 | Very soluble in water | Epilepsy |
| ESTAZOLAM | 8-chloro-6-phenyl-4H-s-triazolo[4,3-□][1,4]benzodiazepine | 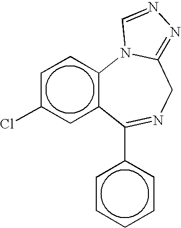 | | Epilepsy |
| PHENOBARBITAL | 5-Ethyl-5-phenylbarbituricacid (C12H12N2O3) | 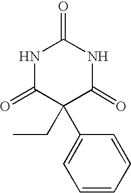 | Very soluble in water | Epilepsy |
| HALAZEPAM | 7-Chloro-1,3-dihydro-5-phenyl-1-(2,2,2-trifluoroethyl)-2H-1,4-benzodiazepin-2-one | 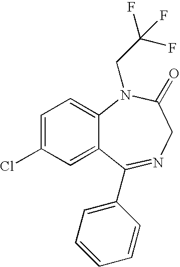 | | Epilepsy |
| KETAZOLAM | 11-Chloro-8,12b-dihydro-2,8-dimethyl-12b-phenyl-4H-[1,3]oxazino[3,2-d][1,4]benzodiazepine-4,7(6H)-dione | 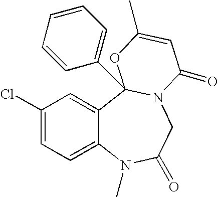 | Practically insoluble inwater; slightly solublein alcohol; freelysoluble in chloroform | Epilepsy |
| QUAZEPAM | 7-Chloro-5-(2-fluorophenyl)-1,3-dihydro-1-(2,2,2-trifluoroethyl)-1,4-benzodiazepine-2-thione. | 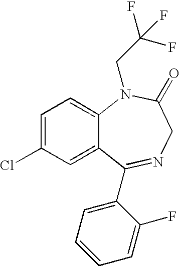 | | Epilepsy |
| PRAZEPAM | 7-Chloro-1-(cyclopropylmethyl)-1,3-dihydro-5-phenyl-2H-1,4-benzodiazepin-2-one | 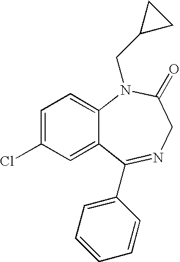 | Slightly soluble inwater; freely solublein chloroform and inmethyl alcohol;practically insoluble inisooctane | Epilepsy |
| TEMAZEPAM | 7-chloro-1,3-dihydro-3-hydroxy-1-methyl-5-phenyl-2H-1,4-benzodiazepin-2-one | 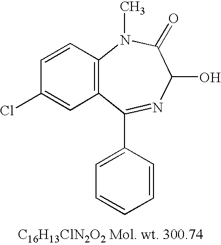 | | Epilepsy |
| NITRAZEPAM | 1,3-Dihydro-7-nitro-5-phenyl-2H-1,4-benzodiazepin-2-one | 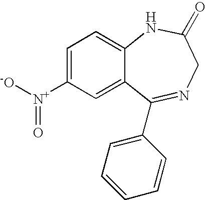 | Soluble 1 in 12 ofwater, 1 in 14 ofalcohol, and 1 in 3.5of chloroform;insoluble in ether;freely soluble inmethyl alcohol | Epilepsy |
| DIAMOX | N-(5-Sulfamoyl-1,3,4-thiadiazol-2-yl)acetamide | 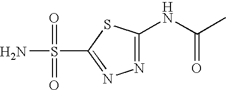 | | Epilepsy |
| CARBATROL | 5H-dibenz[b,f]azepine-5-carboxamide | 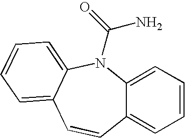 | Slightly soluble inwater and in alcohol | Epilepsy |
| DIASTAT | 7-chloro-1,3-dihydro-1-methyl-5-phenyl-2H-1,4-benzodiazepin-2-one | 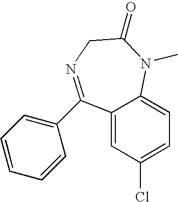 | | Epilepsy |
| FELBAMATE(FELBATOL) | 2-phenyl-1,3-propanedioldicarbamate | 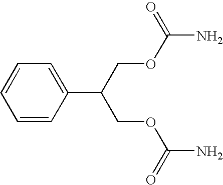 | Freely soluble inwater and in alcohol;soluble in acetone;insoluble in ether andin benzene | Epilepsy |

In some embodiments, the active agents may exhibit increased stability and/or solubility at acid or alkaline pH and may be centrally administered in such form. In other embodiments, a physiologically suitable pH (e.g., in the range of about pH 7.2-7.4) may be preferred for central administration. However, titration to physiological pH may result in solubility and/or stability issues for many active agents. Therefore, it may be preferred in some cases to develop aqueous formulations in which the active agent is formulated with a solubility enhancing agent or stabilizing excipients at a physiologically suitable pH. If titration is desired, any suitable buffer known in the pharmaceutical arts may be used (e.g., phosphate, acetate, glycine, citrate, imidazole, TRIS, MES, MOPS).

Further it may be desirable to maintain physiological isotonicity. For instance, in certain embodiments, an osmolality ranging from about 100 to about 1000 mmol/kg, more particularly from about 280 to about 320 mmol/kg may be desired. Any suitable manner of adjusting tonicity known in the pharmaceutical arts may be used, e.g., adjustment with NaCl.

In accordance with certain aspects of the invention, pharmaceutical compositions are designed to maximize solubility and stability in the CSF and under conditions of use for chronic administration to the CSF. In this regard, it has been found in accordance with the present invention that the maximum aqueous solubility for fat soluble drugs is close to their effective concentrations. For example, in certain embodiments, e.g., when the active agent is felbamate or carbamazapine, the concentration in the formulation must be increased five-fold over the aqueous solubility limit in order to achieve therapeutic concentrations in rat ventricles. For more water soluble drugs, it has been found in accordance with the present invention that the upper limits of tonicity or viscosity in CSF is the maximal possible concentration. For example, valproate can be solubilized up to 50-fold that of the therapeutic concentration, but the solution becomes hypertonic.

2. Solubility Enhancing Agents

Again, in accordance with certain embodiments of the invention, it has been found particularly advantageous to formulate active agents in aqueous solutions at physiological pH and tonicity. However, to provide adequate solubility to the composition, the use of solubility enhancing agents may optionally be required.

Without intending to be limited by theory, in certain aspects, solubility enhancing agents may utilize their amphiphilic characteristics to increase the solubility of active agents in water. As generally understood by those skilled in the art, a wide variety of solubility enhancing agents that possess both nonpolar and hydrophilic moieties may be employed in connection with the present invention. Solubility enhancing agents that are currently employed in parenteral formulations are known to be relatively non-toxic when administered systemically. However, amphiphilic agents possessing stronger hydrophobic character have the potential to interact with cell membranes and produce toxic effects. Therefore, again, without intending to be limited by theory, solubility enhancing agents with minimal hydrophobic character may be preferred in certain embodiments within the context of the present invention, as such agents will be well-tolerated during chronic central administration.

In addition to minimizing the hydrophobic character of the solubilizing agents employed, toxicity during chronic central administration may be reduced if the solubility enhancing agent is readily degraded in a cellular environment. The ability of cells to degrade compounds prevents their accumulation during chronic administration. To this end, the solubility enhancing agents may optionally include chemically-labile ester and ether linkages that contribute to low toxicity, and thereby prevent significant cellular accumulations during chronic central administration.

In this regard, in accordance with certain embodiments of the invention, the solubility enhancing agent may be selected from cyclodextrins, e.g., p-hydroxypropyl-cyclodextrin, sulfobutyl-ether-β cyclodextrin, etc. Previous studies are consistent with this hypothesis and report that beta cyclodextrin had no measurable toxicity when administered intrathecally (Yaksh et al, 1991; Jang et al, 1992).

In other embodiments, the solubility enhancing agent may be selected from sucrose esters. Such agents are formed of two benign components (sucrose and fatty acids) linked by a highly labile ester bond. Although a readily-degradable linkage is beneficial from a toxicity standpoint, the solubility enhancing agent must be sufficiently robust to maintain its ability to solubilize the active agent during the desired conditions of use, e.g., during a suitable duration of time for chronic central administration within an implantable intrathecal device, in the acellular environment.

Generally, certain compositions of the invention may be prepared by formulating the desired amount, which may be a therapeutically effective amount, of the desired active agent in a suitable solubility enhancing agent. Solubility enhancing agents include, but are not limited to, e.g., cyclodextrins, octylglucoside, pluronic F-68, Tween 20, sucrose esters, glycerol, ethylene glycol, alcohols, propylene glycol, carboxy methyl cellulose, solutol, mixtures thereof, etc. Other solubility enhancing agents include, but are not limited to, polyethylene glycol (PEG), polyvinlypyrrolidone (PVP), arginine, proline, betaine, polyamino acids, peptides, nucleotides, sorbitol, sodium dodecylsulphate (SDS), sugar esters, other surfactants, other detergents and pluronics, and mixtures thereof. Alternatively, stable multiphase systems could be employed to safely solubilize therapeutics for intrathecal delivery (e.g., liposomes, micro/nano emulsions, nanoparticles, dendrimers, micro/nano spheres).

Any suitable amount of solubility enhancing agent sufficient to solubilze the active agent of interest to the desired concentration may be used. In certain embodiments, molar ratios of active agent to solubility enhancing agent ranging from about 0.5:1 to about 1:10, particularly, about 1:1 to about 1:5, more particularly 1:1 to about 1:2, may be used to achieve adequate solubility of the active agent to the desired concentrations.

3. Stabilizing Excipients

In addition to solubility, the active agent must be sufficiently stable within the composition to withstand hydrolytic and oxidative degradation in order to maintain biological activity during central administration. While the active agents generally possesses the therapeutic effects observed during conventional administration following injection into the CSF, the stability of the drug in the composition prior to central administration is also of importance. To this end, in certain embodiments, the compositions of the present invention may further include stabilizing excipients and buffers.

Considering that oxidation represents a common degradation pathway, in certain aspects, the compositions of the invention may be deoxygenated (e.g., by saturating with nitrogen gas) to minimize the formation of reactive oxygen species that would degrade the active agent during storage. Another method would be to ensure that formulations are stored in a container that does not allow passage of light, thereby minimizing photo-induced degradation. Clearly, both the removal of oxygen and protection from light can be easily accomplished in a device designed for use in chronic central administration. In addition, in accordance with certain aspects of the invention, stabilizing excipients may optionally be used to, e.g., prevent or slow degradation by oxidation and/or hydrolysis of the active agents. For example, vitamin E, methionine, chelators and mannitol may be used to reduce oxidative degradation. Since the rates of many degradation reactions are pH-dependent, such formulations may include any suitable buffering agent known in the art (e.g., phosphate, acetate, glycine, citrate, imidazole, TRIS, MES, MOPS).

Stabilizing excipients useful in the context of the compositions described herein include any pharmaceutically acceptable components which function to enhance the physical stability, and/or chemical stability of the active agent in the compositions of the invention. The pharmaceutical compositions described herein may include one or more stabilizing excipient, and each excipient may have one or more stabilizing functions.

In one aspect, the stabilizing excipient may function to stabilize the active agent against chemical degradation, e.g., oxidation, deamidation, deamination, or hydrolysis. In this regard, the stabilizing excipients may optionally be selected from antioxidants, such as ascorbic acid (vitamin C), vitamin E, tocopherol conjugates, tocopherol succinate, PEGylated tocopherol succinate, Tris salt of tocopherol succinate, Trolox, mannitol, sucrose, phytic acid, trimercaprol or glutathione.

4. Penetration Enhancing Excipients

The compositions of the invention may further include optional penetration enhancing excipients. Such penetration enhancing excipients may include any pharmaceutically acceptable excipient known in the art which is capable of maintaining the active agent within the CSF, or otherwise maximizing the active agents residence time in the CSF. In certain aspects, such excipients may act to decrease drug resistance. For instance, the penetration enhancing excipients may act to avoid, bind, or otherwise mask glycoprotein pumps which act to clear the active agents from the CSF. Again, any suitable excipient capable of maintaining the active agent in the CSF, or otherwise maximize CSF residence time may be used.

5. Exemplary Compositions

In certain embodiments, the active agent may be clozapine, felbatol, adenosine (and analogues thereof, e.g., a1 and a2 agonists, a1 and a2 analogue agonists, etc.), lamictal, bumex, valproate, or tegretol (or combinations thereof), and may be solubilized in saline at pH 7.4 by including various optional solubilizing agents/stabilizing excipients in the formulation. In certain embodiments, compositions of such active agents will remain in solution and maintain chemical integrity (e.g., less than about 10% degradation, less than about 5% degradation, less than about 2% degradation, etc.) for at least three months at physiological temperatures (e.g., about 37° C.), thereby providing suitable formulations for chronic central administration in accordance with certain aspects of the invention.

By way of non-limiting example, mass spectrometry may be utilized to assess the chemical stability of the active agent in the composition under conditions to simulate chronic central administration. By way of non-limiting example, such conditions include, e.g., physiological pH at about 37° C. for at least about 3 months, at least about 4 months, at least about 5 months, at least about 6 months, etc.

### B. Central Administration

Another aspect of the present invention relates to central administration in the treatment of CNS-related conditions and disorders. Without intending to be limited by theory, it has been found that central administration, e.g., local delivery to the cerebrospinal fluid (CSF), cerebral ventricles, etc., in accordance with the present invention provides for, e.g., improved bioavailability, reduced systemic toxicity, improved patient compliance, and facilitates complex dosing regimens. Any suitable manner of central administration known in the art may be used, e.g., intrathecal delivery, intrathecal (administration into the cerebrospinal fluid-containing space), including spinal or lumbar delivery into the subarachnoid space; intracranial delivery (administration into the brain parenchyma); intracerebroventricular (ICV) delivery, (administration into the cerebral ventricles), etc.

The central administration may be acute or chronic, and may be via injection, infusion, pump, implantable pump, etc. In certain preferred embodiments, the central administration is via an implantable pump, e.g., an ICV or subarachnoid delivery device for chronic administration. By way of non-limiting example, devices such as those disclosed in U.S. Patent Publication 2004/0133184, which is herein incorporated by reference, may be used.

In this regard, advantages which have become apparent with chronically spinally-administered opiates include local administration in the spinal cord region where the medications mediate their effect, an increase the bioavailability of those medications, and an ability to facilitate therapeutically difficult medications getting to the appropriate spinal cord areas of activity (Yaksh et al. 1999). These advantages and others have also been found to apply to chronic central administration of CNS-active therapeutic agents in accordance with the present invention.

By way of background, in a series of experiment from the 1970s, medications were administered ICV and in the spinal axis acutely. Small molecules (amino acids, chemotherapeutic agents and nucleic acid analogs) were injected ICV and pain medications were injected into the spine. A primary finding from those studies is that the degree of hydrophobicity in a compound's structure predicted biodistribution (amount distributed and rate of distribution) of opiate active medications into the central nervous system parenchyma when medications are administered directly into the CSF. (Balis et al. 2000, Blasberg et al. 1975, Ghersi-Egea et al. 1996, Grossman et al. 1989, Herz et al. 1970, Kessler et al. 1976). Subsequent experience has given support to those original insights and provided basis for the extensive testing and development of spinally-administered medications. Furthermore, methods of quantifying how fast and how far the medications penetrated into the brain were developed (Blasberg et al. 1975, 1977, Collins et al. 1983) and are the basis for the “coefficient of penetration” to understand how much drug is getting into the tissue of interest.

There is limited data in humans related to ICV administered medications for psychiatric disease in terms of how these medications permeate into the brain, and at what rate it occurs (Campbell et al. 1988, Urca et al. 1983). Clinical experience with other medications has been limited to intermittent single bolus injection primarily for infection. (Pickering et al. 1978). However, in accordance with certain embodiments of the present invention, it was found that chronic ICV administration exhibits superior therapeutic results.

In accordance with certain aspects of the invention, establishing drug efficacy in the central nervous system through central administration may be maximized using several strategies. First, certain CNS-active therapeutic agents are likely to be more ideally suited to administration into the ventricle of the brain or the cistema magna than into the spine. For instance, antidepressants, antiepileptics and antipsychotics likely need greater exposure to the brain in the cranium than via the spinal canal which likely is better for certain types of chronic pain and spasticity. Second, certain diseases and disease states would benefit the most by tighter control of dosing regimens for CSF delivery. An example of this is that Parkinson's disease might benefit from multiple times a day administration with a drug holiday. Another example is epilepsy, where administering the active agent before waking would eliminate a patient's seizures that occur on waking in the morning. Women who have seizures at their menstrual period could be given higher level of medication for the 5-7 days around their period than at other times of the month, to maximize medication efficacy. Some drugs may also work reasonably well with lumbar spinal administration but there will be an incremental decline in efficacy relative to application above the cisterna magna.

Dosing strategies also will incorporate various approaches to initiating treatment, stopping treatment, switching treatment and responding to different patient states for central fluid administration. These various dosing strategies can be selected by a manual adjustment of a computer program and/or algorithm. Different initiating treatments include rapid initiation, moderate initiation or slow initiation. Altered initial dosing patterns may be necessary due to such issues as central side effect profiles which may necessitate slower loading (e.g. sedation with quetiapine) or acute suicidality might require rapid initiation (e.g. atypical antipsychotics in a bipolar patient who is suicidal). Patients with this approach may differ because of the central side effect profile which may necessitate slower loading (e.g. sedation with quetiapine) or patients with acute suicidality might require rapid initiation (e.g. atypical antipsychotics in a bipolar patient who is suicidal). Patients may need to have rapid or slow medication taper depending on side effect issues and patient safety. Reasons for performing a rapid taper include reacting to a medication allergy or cross-taper with initiation of another treatment. One Reason for a slow taper might be mediate seizures that caused by rapid withdrawal. Certain reasons to initiate special approaches to treatment might be seizures where a family member or patient might wish to give extra doses for auras or ongoing seizure where an extra dose of medication should appropriately be applied. Tardive Dyskinesia is a side effect syndrome that is believed to be related to dopamine receptor binding above 70% and antipsychotic efficacy occurs with binding above 60% so creating a steady state between 60 and 70% receptor binding. This spectrum of receptor binding is likely also important in other CNS diseases.

Examples of manual or programmed dosing modes or strategies for spinal fluid injected medication include night time administration, administration before waking, increased administration one week a month, three times a day, continuous dosing, bolus dosing, taper dosing, need based dosing, feedback dosing by the physician, provider, patient or family. The clinical scenarios where these can be employed include chronic disease, disease exacerbation, need for suppression treatment, need for recurrence treatment, or state treatment like mania, increase in frequency of seizures or increase in suicide attempts.

Toxicity due to local delivery to the CNS is more complex because of direct administration and more varied ways of medication administration. It follows directly after drug efficacy. The first concept is the concept related to drug level. Antipsychotics are an example of this problem and that levels of medication which cause receptor occupancy above 85% induce drug side effects and above 65% induce beneficial drug effects in the patient population. A solution to this problem is to use computer programming to identify a precise dosing amount that is within this therapeutic window. This amount could be determined by clinical response and complaints, electrophysiological tests like EEG, EP or MEG or by scanning like MRI and PET scanning.

Another problem with long term administration is total dosing wherein drug toxicity is cumulative. An example is the chemotherapeutic methotrexate that can cause severe and potential lethal changes in the glial cells if too much is administered over time. Solutions include limiting the total amount of drug delivered by strictly limiting the dosing period, reducing the dosage, or potentially taking a drug holiday.

A third issue that comes up in toxicology has to do with local drug effects of the medication and its accompanying excipient. Medications administered into the fluid around the brain might be more toxic in the fluid above the spinal cord than if administered in the ventricle. An example of this is that an excipient which might be administered in a 20% concentration in the pump might be able to be diluted 1000 fold in the ventricle versus 10 fold in the spinal fluid because of the relatively different volumes in the spinal cord area (approximately 100 micro liters) versus in the ventricle (approximately 7 cc). Solutions to this dilution problem would present themselves by administering the medication in the ventricle or in the cistema magna if a greater amount of fluid is required for more complete dilution.

Another facet of local drug effect is pH. Available data suggests that it is safe to inject a small amount of weakly buffered or unbuffered, very low pH drug (pH 2.0). An example of this is clozapine that can be solubilized at pH 2.0 and injected safely into the human ventricle. However, some minimal buffering capacity is advantageous to maintain pH-dependent solubility in the pump reservoir. This is counterintuitive to many experts who would assume that normal pH is a requirement of intra CSF administration.

Toxicology experiments can be constructed in vitro and in vivo to prepare for medications administered in the CSF. Initial in vitro toxicology work for CSF based-drug delivery involves testing whether medication/excipient combinations cause cell death, oxidation or other metabolic changes. In vitro experiments ideally are performed in two animal species such as the rat and the dog. The rat is a good for preliminary testing because of availability of dosing to 28 days but the volume of the ventricle is very small and therefore less dilution will occur than in human ventricular delivery. The dog offers the capacity for 90 day drug testing using an implanted catheter and a pump that is carried on the animal's body.

In this regard, it has been found in accordance with certain embodiments that the activity of certain CNS-active agents is substantially local to the delivery site within the CSF. Bernards et al. (2006) studied slow drug administration into the spinal CSF and found that both hydrophobic and hydrophilic compounds bind within ˜1 cm of the local area of drug administration. In addition, CSF flow from the lumbar cistern differs from supratentorial CSF flow in that it tends to be slower, and likely does not go through the ventricles or equilibrate with supratentorial CSF compartments (Kroin et al. 1993). As such, without intending to be limited by theory, the central administration delivery device may be advantageously placed in close proximity to the location of therapeutic activity for the target CNS condition or disorder for treatment.

With regard to the treatment of schizophrenia with clozapine, the hippocampus, basal ganglia and neocortex are the brain areas that show clozapine binding in the CNS, and they are relatively remote to the lumbar cistern (Nordstrom et al. 1995). As such, in one embodiment, the mode of central administration for the treatment of schizophrenia with clozapine may preferably be ICV administration. Similarly, for the treatment of epilepsy, MS, etc., the mode of central administration may preferably be ICV administration.

### C. Methods of Use

In another aspect, methods of using the compositions described herein are provided. The methods generally comprise centrally administering a formulation described herein to a subject in need thereof. The methods can be used in any therapeutic or prophylactic context in which the active agent may be useful. By way of non-limiting example, the methods may include treatment of a variety of CNS conditions, including but not limited to Alzheimer's disease, dementia, anxiety, schizophrenia, pain, drug addiction, bipolar disorder, anxiety, major depressive disorder (MDD), depression, sleep disorders, encephalitis, multiple sclerosis (MS), closed head injury, Parkinsons disease, Tourette's Disorder, brain tumors and epilepsy, or any other known use of disclosed active agents. Yet other aspects of the invention include the treatment and prevention of addiction and related disorders, as well as obesity.

In accordance with the methods disclosed herein, a pharmaceutical composition may be centrally administered in any manner known in the art such that the active agent is biologically available to the subject or sample in effective amounts. For example, IT (intrathecal) administration, spinal administration, ICV (intracerebroventricular), etc. delivery may be used. Determination of the appropriate administration method is usually made upon consideration of the condition (e.g., disease or disorder) to be treated, the stage of the condition (e.g., disease or disorder), the comfort of the subject, and other factors known to those of skill in the art.

Administration may be intermittent or continuous, both on an acute and/or chronic basis. Continuous administration may be achieved using an implantable or attachable intrathecal pump controlled delivery device, such as those marketed by Medtronic, Inc. However, any implanted controlled delivery device known in the art may be used.

Certain embodiments involve using an implanted catheter pump system for at least one month, at least about two months, at least about three months, at least about 4 months, at least about 5 months, at least about 6 months, etc. of chronic central administration, e.g., ICV.

In one embodiment, administration can be a prophylactic treatment, beginning concurrently with the diagnosis or observation of condition(s) (e.g., lifestyle, genetic history, surgery, etc.) which places a subject at risk of developing a specific disease or disorder. In the alternative, administration can occur subsequent to occurrence of symptoms associated with a specific disease or disorder.

In one embodiment, the present invention relates to the treatment of patients with a CNS condition or disorder comprising centrally administering a composition comprising an agent active in the treatment of said CNS condition or disorder. In certain aspects, the agent is administered ICV over a predetermined duration of time, and the composition is formulated so as to maintain solubility and stability over the predetermined time period and conditions of use (e.g., physiological pH, temperature, and/or tonicity, etc.). The duration of time may be, e.g., at least about 1 month, at least about 2 months, at least about 3 months, at least about 4 months, at least about 5 months, at least about 6 months, etc. In addition, the ICV administration may be accomplished via an implantable intrathecal pump. In certain embodiments, the CNS condition or disorder may be, e.g., epilepsy, schizophrenia, anxiety, depression (or related disorders), MS, etc. Further, the active agent may be, e.g., felbatol or adenosine (epilepsy) clozapine (schizophrenia), phenelzine or adenosine (anxiety or depression) etc.

In another embodiment, the present invention also relates to the treatment of patients with multiple sclerosis with an implantable intrathecal pump and with use of reformulated small molecules including all non steroidals (of which indomethacin is an example), all steroids (of which prednisone is an example), methotrexate, cyclosporine, antcyclosporine, indomethacin, etc. for long-term chronic treatment and disease control. The medication treatment for MS can also be treatment for CNS viral encephalitis on both a chronic and acute basis.

The term “effective amount” refers to an amount of an active agent used to treat, ameliorate, prevent, or eliminate the identified CNS condition (e.g., disease or disorder), or to exhibit a detectable therapeutic or preventative effect. The effect can be detected by, for example, chemical markers, antigen levels, or time to a measurable event, such as morbidity or mortality. The precise effective amount for a subject will depend upon the subject's body weight, size, and health; the nature and extent of the condition; and the therapeutic or combination of therapeutics selected for administration. Effective amounts for a given situation can be determined by routine experimentation that is within the skill and judgment of the clinician.

For any active agent, the effective amount can be estimated initially either in cell culture assays, e.g., in animal models, such as rat or mouse models. An animal model may also be used to determine the appropriate concentration range and route of administration. Such information can then be used to determine useful doses and routes for administration in humans.

By way of non-limiting example, in certain embodiments, exemplary Effective Daily Doses for ICV (animal) compared with oral (human) for various CNS-related conditions and disorders is provided in the table below. The indicated % of Oral dose is indicative of the difference in effective dosages between systemic administration and central administration, as well as the impact on systemic exposure following central administration (thereby reducing toxicity, etc.). As such, in certain embodiments, the centrally administered dosage may range from about 0.4% to about 225% of the corresponding systemic administration dosage. In some embodiments, the centrally administered dosage is from about 30-fold lower to about 30-fold higher than the ICV dose in rodents, e.g., the centrally administered dosage in humans is about 30-fold lower to about 30-fold higher than the effective ICV dose in rats. For example, in some embodiments, the dosage is about 30-fold lower to about 30-fold higher than the dose listed in the below table for particular drugs for rodent ICV dose. In some embodiments, the centrally administered dosage is about 20-fold lower to about 30-fold higher, about 10-fold lower to about 30-fold higher, about 10-fold lower to about 20-fold higher, about 10-fold higher to about 10-fold higher, about 10-fold higher, about 20-fold higher, about 30-fold higher, or about 10-fold lower, about 20-fold lower, about 30-fold lower.

| | | Human oral | | |
|---|---|---|---|---|
| | Rodent ICV | equivalent | % of Oral | |
| Active Agent | dose (mg/kg) | (mg/kg) | dose | Efficacy |
| Felbamate | 0.08242 | 20 | 0.4% | effective in blocking |
| | | | | seizures |
| Adenosine* | 0.001336 | 0.1 | 1.3% | effective in blocking |
| | | | | seizures |
| Lamictal | 0.00256 | 3.33 | 0.07% | effective in blocking |
| | | | | seizures |
| Bumex | 0.000364 | 0.033 | 1.1% | effective in blocking |
| | | | | seizures |
| Valporate | 0.1442 | 1 | 14.4% | effective in blocking |
| | | | | seizures |
| Tegretol | 0.00888 | 6.66 | 0.13% | effective in blocking seizures |
| Clomiparmine | 0.37 | 4.16 | 8.8% | anxiety reduction |
| Phenelzine | 1.8 | 1.5 | 120% | anxiety reduction |
| Fluoxetine | 0.74** | 0.33 | 224% | anxiety reduction |
| Tranylcypromine | 0.0319** | 1 | 31.9% | anxiety reduction |
| Clozapine | 0.040 | 7.5 | 0.5% | acute effective dose for |
| | | | | Improvement of sensory |
| | | | | inhibition deficits in mice |

*human dose is IV not oral for this drug
**estimated effective dose

To evaluate the efficacy of the methods of the present invention in the treatment of schizophrenia, the DBA/2 mouse (Stevens et al, 1996) described in further detail in the examples below may be used as a model for the sensory inhibition deficits in schizophrenia. The DBA/2 mouse bears both genotypic as well as phenotypic similarities to schizophrenia with regard to sensory inhibition. Studies of the DBA/2 and C3H strains of mice have identified a restriction fragment length polymorphism (RFLP) in the α7 receptor between the two strains (Stitzel et al 1996) which parallels the findings of polymorphisms in the human CHRNA7 from schizophrenia patients (Freedman et al 1997). Recent studies have demonstrated polymorphisms in the promotor region of the a gene in humans (Leonard et al 2002) and DBA/2 mice (Stitzel et al 2003). It is postulated that these polymorphisms in gene coding in humans and DBA/2 mice may underlie the roughly 50% reduction in the numbers of hippocampal α7 nicotinic receptors observed in both schizophrenia patients (Breese et al 1997) and DBA/2 mice (Stevens et al 1996). These reductions are thought to underlie the deficits in sensory processing observed (Freedman et al 1995; Stevens et al 1996). In certain embodiments, to evaluate epilepsy can be done using several models of epilepsy including the acute PTZ model, carotid ligation and Kainate. We demonstrated that using acute PTZ model demonstrated alteration of the seizure threshold.

In certain embodiments to evaluate depression and anxiety there are animal models including elevated plus, open maze, water tank. We demonstrated that alteration of time in the elevated plus open arm and open maze showed efficacy for reformulated antidepressants and antianxiety agents. Such behavioral paradigms can demonstrate decreased anxiety by increased entry into the open arms of the elevated plus maze, and increased activity in the central areas of the open field maze (Mechiel Korte and De Boer 2003; Crawley 1985). Both the open field and elevated plus mazes can demonstrate increased generalized activity levels by showing increased distances traveled over a give time period, or sedation by decreased distances traveled. The swim tank can show decreased behavioral despair (interpreted to represent depression) by increased struggling to escape the water (Russig et al 2003).

In certain embodiments, other types of model systems may be utilized to determine the efficacy, stability, toxicity and other pharmacologic or pharmacokinetic properties of CNS active agents administered by ICV. For example, closed head injury and/or spinal cord injury may be modeled by using a pneumatic or controlled weight impact (New York Impactor) injury to exposed animal spinal cords, followed by ICV administration of various agents. Alternatively, spinal cord transaction, cortical contusion, impact acceleration or fluid percussion may also be used to model such injuries.

In other embodiments, multiple sclerosis may be modeled by experimental allergic encephalomyelitis (EAE), adjuvant arthritis, Theiler's murine encephalomyelitis virus (TMEV), or mouse hepatitis virus (MHV) infection. Stroke may be modeled by middle cerebral artery occlusion. Parkinson's disease may be modeled by reserpine-induced dopamine depletion, chemical or electrical lesion, or administration of 6-OHDA or MPTP. MAOs have been shown to work in Parkinson's disease and we demonstrate MAOs can work in the anxiety and depression models discussed above.

In other embodiments for bipolar disorder, clozapine which has been shown to be effective clinically for schizophrenia is also effective for bipolar disorder. This has been tested as such in our initial schizophrenia data already discussed.

Alzheimer's disease may be modeled using known transgenic mouse model systems. Huntington's disease may be modeled using GABAnergic lesions with antagonists or using NMDA aganoists. Alternatively 3-nitropropionic acid may be administered to animal models to create a permanent Huntington's like condition. Epilepsy may be modeled using generalized seizure models with DBA/2 mice, genetically epilepsy prone rats or gerbils, maximal electroshock models, simple parietal seizure models such as with microapplication of convulsant drugs, penicillin, picrotoxin, bicuculin, strychnine or kainic acid. Chronic seizure models such as by application of alumina hydroxide, cobalt, tungsten or zinc. Or complex parietal seizure models as by injecting tetanus toxin into the hippocampus.

Model systems for anxiety include fear-potentiated startle reflex, conflicts test (food in open field, Vogel punished drinking), an elevated plus maze, social interaction or approach/avoidance paradigm. Depression may be modeled with Porsolt (forced) swim, tail suspension, olfactory bulbectomized rats, Flinders Sensitive Line rates, Fawn Hooded rats, learned helplessness or maternal separation. Anhedonia may be modeled using novelty object place conditioning. Model systems for drug addiction include any chronic drug exposure model (inhalation, continuous perfusion, repeated injection, self-administration).

In yet another embodiment, the methods disclosed herein further comprise the identification of a subject in need of treatment, particularly a subject refractory to standard systemic administration of CNS-active agents. In more particular embodiments, patients who have failed two or more standard systemic therapies or whose conditions are severe enough to warrant more aggressive treatment than standard systemic therapies may benefit from intrathecal delivery. Any effective criteria may be used to determine that a subject may benefit from administration of CNS-active agent. Methods for the diagnosis of CNS-related conditions and disorders, for example, as well as procedures for the identification of individuals at risk for development of these conditions, are well known to those in the art. Such procedures may include clinical tests, physical examination, personal interviews and assessment of family history.

### D. Kits

Also provided are kits for central administration of the formulations described herein.

In certain embodiments the kits may include a desired amount of at least one pharmaceutical formulation as disclosed herein. Kits may further comprise suitable packaging and/or instructions for use of the formulation. Kits may also comprise a means for the delivery of the formulation, for example, for delivery to the receptacle of a pump. Kits may also comprise a receptacle of the pump that contains the formulation in the appropriate amount and concentration. Other devices that are used as part of a system for central administration are known to those of skill in the art and these may be included as part of a kit.

The kits may include other agents for use in conjunction with the therapeutic agents in the formulations for central administration for CNS conditions. These other agent(s) may be provided in a separate form, or mixed with the therapeutic agents for CNS conditions, provided such mixing does not reduce the effectiveness of the agent or formulations described herein and is compatible with central administration.

The kits may include appropriate instructions for preparation and administration of the formulation, side effects of the formulation, and any other relevant information. The instructions may be in any suitable format, including, but not limited to, printed matter, videotape, computer readable disk, or optical disk.

In another aspect of the invention, kits for treating an individual who suffers from the conditions described herein are provided, comprising a first container comprising the appropriate dosage amount of a formulation described herein, and instructions for us. The container may be any of those known in the art and appropriate for storage and delivery of the formulations. In certain embodiments, the kit further comprises a second container comprising a pharmaceutically acceptable carrier, diluent, adjuvant, etc. for preparation of the composition to be administered to the individual.

Kits may also be provided that contain sufficient dosages of the formulations as disclosed herein to provide effective treatment for an individual for an extended period, such as 1-3 days, 1-5 days, a week, 2 weeks, 3 weeks, 4 weeks, 6 weeks, 8 weeks, 3 months, 4 months, 5 months, 6 months, 7 months, 8 months, 9 months or more.

Kits may also include multiple doses of the formulations and instructions for use and packaged in quantities sufficient for storage and use in pharmacies, for example, hospital pharmacies and compounding pharmacies.

To assist in understanding the present invention, the following Examples are included. The experiments described herein should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof.

### Example 1

### Exemplary Compositions

### A. Clozapine

Clozapine is an organic compound that is “practically insoluble” in water. In accordance with certain aspects of the invention, “practically insoluble” includes agents that dissolve at a concentration of less than about 0.01%. This low solubility is reflected by its high octanol-to-water partition coefficient of 1000 at pH 7.4 (Merck Index, 2004). This value indicates that clozapine is one thousand times more soluble in organic solvents (i.e., octanol) than in water at pH 7.4. However, the value for the partition coefficient is lowered dramatically under acidic conditions (0.4 at pH 2), demonstrating that the drug can be solubilized at low pH. Considering that clozapine has two titratable groups with pKₐs of 3.7 and 7.6, it is not surprising that acidic conditions protonates the molecule and produces a cationic form that is freely soluble in water. Thus, when clozapine is added to water that has been acidified with HCl, a clear yellow solution forms that has minimal absorbance from 400-800 nm. Progressive addition of NaOH steadily increases the pH of the clozapine solution with little effect on solubility until approximately pH 6.5. As neutral pH is approached, precipitation of clozapine is dramatic, and results in a sharp increase in the absorbance at 500 nm due to the presence of insoluble drug particles.

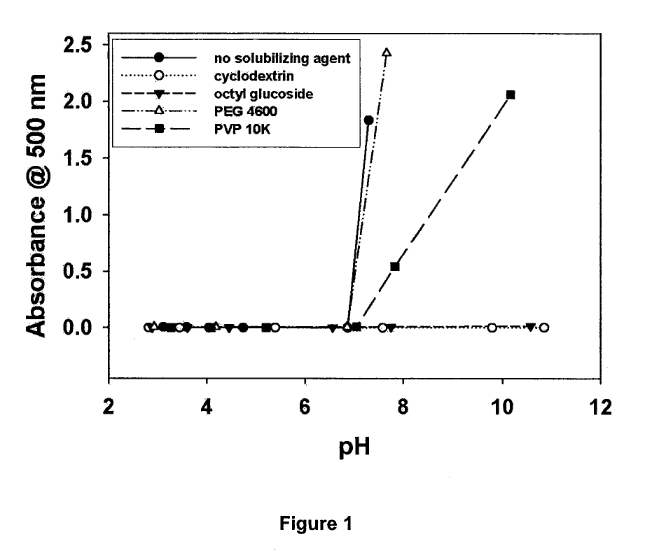

As shown in FIG. 1, it was unexpectedly found in accordance with certain aspects of the invention that polyethylene glycol (PEG 4000) and polyvinylpyrrolidone (PVP 10K) were not able to prevent clozapine precipitation as the solution was titrated above neutral pH. In contrast, both cyclodextrin and octyl glucoside prevented clozapine precipitation even at very alkaline pH (≈11), indicating that both of these compounds serve as potent solubility enhancing agents for active agents such as clozapine. Additional experiments have shown that the clozapine remains solubilized at physiological pH for at least two months when stored at 37° C. With reference to FIG. 1, Clozapine was initially solubilized at pH≈3, and the solution was titrated to higher pH. Precipitation of clozapine is indicated by the sharp increase in turbidity (as indicated by enhanced absorbance at 500 nm). Notice that while polyethylene glycol (PEG 4600) and polyvinyl pyrrolidone (PVP 10K) have minor effects on the solubility at higher pH, cyclodextrin and octyl glucoside completely inhibit precipitation of clozapine even at strongly alkaline pH.

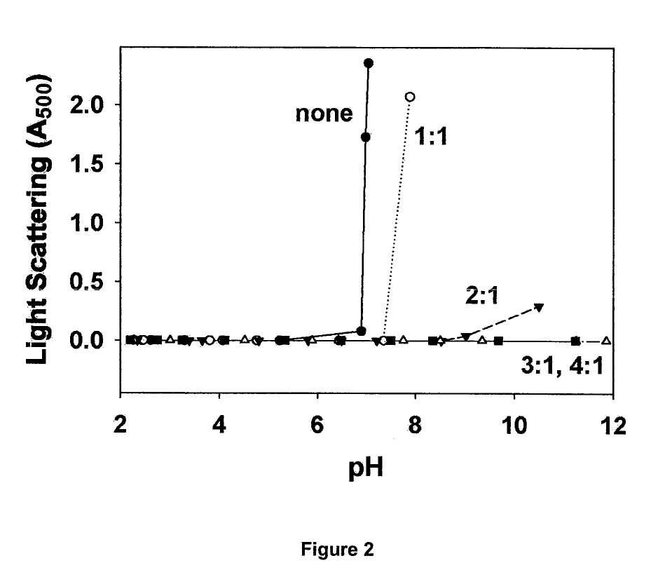

In addition, in accordance with other aspects of the invention, it was found that alteration of the solubilizing agent:active agent ratio was a results oriented parameter in developing soluble formulations. For instance, FIG. 2 shows results from experiments at different cyclodextrin-to-clozapine molar ratios, and demonstrates that a 3:1 ratio is necessary to prevent clozapine precipitation at strongly alkaline pH (≈11), but a lower ratio (2:1) may be capable of maintaining solubility at pH 7.4. With reference to FIG. 2, precipitation of clozapine at high pH is progressively inhibited by the presence of higher molar ratios of cyclodextrin. Although a molar ratio of 2:1 is sufficient to inhibit clozapine precipitation up to pH 9.0, higher levels of cyclodextrin are capable of completely inhibiting precipitation at strongly alkaline pH (>10.0).

These results demonstrate that clozapine can be readily solubilized by solubility enhancing agents that are commonly employed in pharmaceutical formulations for parenteral administration (e.g., cyclodextrin). Due to their use in parenteral formulations, these agents are considered to be relatively non-toxic, at least when delivered systemically. Tween 20 and pluronic F-68 (other commonly employed solubilizing agents) have effects similar to cyclodextrin, and additional solubilizing agents (e.g., sucrose esters) may also be used.

Additional active agents have been similarly formulated, as described in the Examples below.

### B. Stability of Clozapine

Compositions designed for chronic administration via an implanted injection device are exposed to body temperature for an estimated three months before the device is refilled with a fresh solution. During this period, the active agent must remain soluble and resist degradation in order to maintain its biological activity upon injection into the CSF. Therefore, the stability of active agent in compositions of the present invention incubated at 37° C. for a three month period have been examined.

Aqueous formulations (1 mg clozapine/mL in glass vials, pH=7.4) containing clozapine solubilized with beta-cyclodextrin, octyl glucoside, pluronic F-68, Tween 20, or sugar esters are adjusted to isotonicity with NaCl and incubated in the dark at 37° C. for three months. Triplicate samples are examined at 1, 2, and 3 months by UV-Visspectroscopy to assess whether precipitation has occurred (as indicated by A₅₀₀). In addition, studies have shown that clozapine degradation results in absorbance changes in the UV region (Hasan et al., 2002), so an aliquot of each sample is diluted to 0.02 mg/mL and used to assess changes in the UV absorbance profile (200-400 nm). Formulations that maintain clozapine solubility and have UV absorbance profiles identical to fresh controls are further analyzed by mass spectrometry to determine if the molecular weight of clozapine molecules has been altered by hydrolysis or oxidation. At pH 7.4, it is unlikely that hydrolytic reactions will contribute significantly to degradation, and thus we expect that oxidation of clozapine to clozapine-N-oxide will be the major degradation pathway (Lin et al., 1994). Experiments to date have shown that isotonic clozapine preparations solubilized with cyclodextrins and formulated in weak phosphate buffer (10 mM) at physiological pH retain their UV absorbance profile for 2 months at 37° C.

Clozapine analysis is done using a validated LC/MS/MS assay modified from a previously published method (Aravagiri and Marder, 2001). Briefly, 100-200 μl samples are extracted in 10× volume of ethyl acetate:pentane (1:1) containing 1% (v/v) 30% NH₄OH following the addition of 50 ng trazodone (internal standard). Samples are vortexed for 5 minutes, centrifuged and the organic phase collected and dried down with a rotary evaporator. The dried down samples are resuspended in mobile phase (60 mM ammonium acetate (pH 7), methanol and acetonitrile (5:45:50, v/v/v) and analyzed by LC/MS/MS. Samples are analyzed with a PE Sciex API-3000 triple quadropole mass spectrometer (Foster City, Calif.) with a turbo ionspray source interfaced to a PE Sciex 200 HPLC system. The mobile phase is isocratic at a flow rate of 200 μl/min using a C₁₈, 150×2 mm column. Samples are quantitated by internal standard reference in multiple reaction monitoring (MRM) mode by monitoring the transition m/z 327→270 for clozapine and the transition m/z 372→176 for the internal standard (trazodone).

The data showed no change in UV absorbance or detectable precipitation for at least 4 months at 37 degrees.

### C. Toxicity of Cyclodextrin and Clozapine

In addition, a preliminary assessment of the toxicity of cyclodextrin in primary mouse cortical neuron cultures was performed. Primary cortical cultures were obtained from fetal (E15) C57BL/6J mice as previously published (Donohue et al 2006). After dissection, and cellular dissociation, the cells were washed with Dulbecco's Modified Eagle's Medium with 10% fetal bovine serum. Following recentrifugation, the cells resuspended in plating medium, counted with trypan blue and plated at a constant density of 6.5×10⁴ cells per well in a 96-well pre-coated plate. The plating media was 2% B27, 0.5 mM L-glutamine and 25 μM glutamic acid in NEUROBASAL medium (Invitrogen). On the 4^{th} day, half of the medium was replaced with fresh medium that did not contain glutamic acid. The cultures were maintained at 37° C. in a humidified atmosphere of 5% CO₂. On the 7^{th} day of culture incubation, one half the media (40 μL) was replaced with media containing various concentrations of the clozapine-cyclodextrin formulation or cyclodextrin alone. The cultures were incubated and cell toxicity assayed at 24, 48 and 72 hours. Viability was assessed by the MTT (3-(4,5 diethylthiazol-2-yl)-2,5) diphenyltetrazolium bromide) assay, CellTiter 96 Non-radioactive cell Proliferation Assay (Promega, Madison Wis.) and by visual examination.

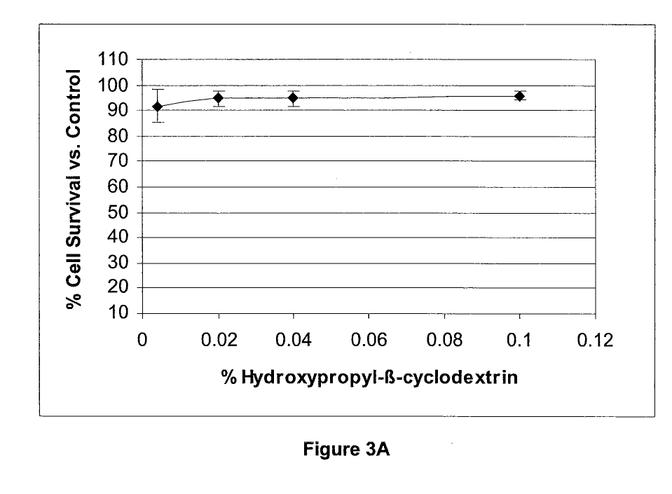

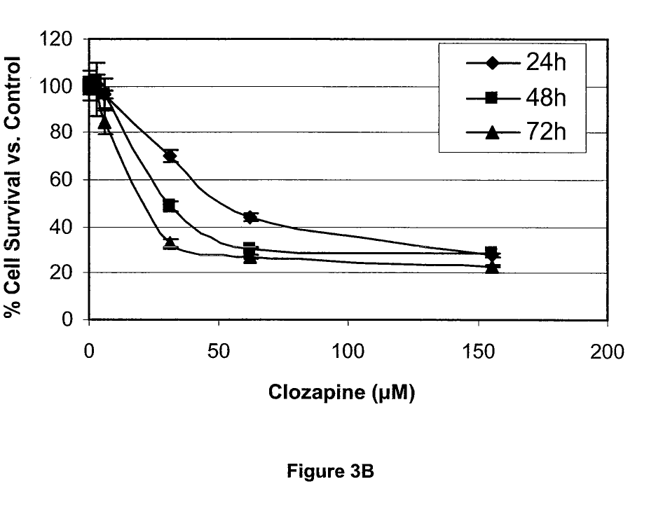

As shown in FIGS. 3A and 3B, the data demonstrate that toxicity is not observed until 10 μg/ml; approximately 100-fold higher than that needed for therapeutic efficacy. Furthermore, cyclodextrin alone exhibited no toxicity, consistent with previous reports (Yaksh et al. 1991, Jang et al., 1992). With reference to FIG. 3A, cell viability is demonstrated at 24 hours at dilutions of cyclodextrin in culture media from 0.002% to 0.1%. There was no significant reduction in neuronal viability with the cyclodextrin solutions which were used to solubilize clozapine. When clozapine was formulated with cyclodextrin and added to the cells, significant toxicity was observed at final concentrations of 10 μg/mL or 30 μM or higher (FIG. 3B). This toxicity is similar to that reported to occur to human neutrophils and monocytes (Gardner et al. 1998).

D. Formulations with Other Active Agents

Other active agents described herein may be solubilized in a manner similar to that described above with regard to clozapine. For instance, the active agent may be solubilized with a solubility enhancing agent such as a cyclodextrin, and pH may be adjusted using, e.g., a phosphate buffer, and the composition made isotonic with, e.g., NaCl.

In accordance with certain embodiments of the invention, compositions including clonidine hydrochloride, trans-2-phenylclyclopropyl-amine hydrochloride, felbamate, and adenosine were prepared at pH 7.4. In other embodiments, compositions including amitriptyline hydrochloride, clomipramine hydrochloride, and imipramine hydrochloride were prepared by solubilizing the active agent in cyclodextrin at active agent:cyclodextrin ratios of 1:1, 1:2, and 1:1, respectively, and adjusting the pH to 7.4 with 10 mM sodium phosphate buffer. Additional examples of compositions prepared in accordance with the present invention are detailed in the examples below.

### Example 2

### Efficacy of ICV Administration

In accordance with certain aspects of the invention, in order to determine if ICV administration of compositions of the invention would treat CNS-related conditions and disorders, the following experiments were designed and/or performed.

### A. ICV Administration of Clozapine, Ondansetron

In order to determine if ICV administration of clozapine would improve sensory inhibition in a manner similar to systemically administered clozapine (Simosky et al 2002), DBA/2 mice were recorded before and after ICV administration of 1 μl of saline containing either 0.5 or 1 μg of clozapine at pH 4.5. The following methods were used to record the sensory inhibition. By way of background, schizophrenia usually presents with a constellation of symptoms which include positive symptoms, negative symptoms, and cognitive deficits (Waterworth et al 2002). Poor inhibitory processing of sensory information is also associated with schizophrenia (Freedman et al 1987) and has been postulated to produce an overload of incoming sensory information such that the individual is “flooded” with input. The flooding then leads to personality decompensation and psychosis (Venebles 1964; 1992).

Specifically, the sensory processing deficit is a failure of sensory input to initiate activity in an inhibitory circuit. Normally, this circuit would be activated by incoming sensory information. The circuit normally remains active for at least 500 msec, such that, if a second identical stimulus arrives, there is partial inhibition of the response. This protects the brain from having to process excessive, repetitive sensory information. Several studies have correlated the severity of sensory inhibition deficits with certain positive symptoms in schizophrenia patients. Specifically, the severity of magical ideation and unreality symptoms are correlated with deficits in sensory inhibition (Croft et al 2001). Other studies have identified a correlation between sensory inhibition deficits and negative symptoms, particularly on indices of impaired attention (Erwin et al 1998). Finally, improvements in sensory inhibition have been correlated with improvement in symptomatology (Nagamoto et al 1999).

P₅₀ sensory inhibition is a measure of adequate inhibitory circuitry which functions to protect an individual from sensory overload. Clinical improvement in schizophrenia has been shown to directly correlate with improvement in P₅₀ sensory inhibition in humans with adequate dosage of clozapine (Nagamoto et al 1999). P₅₀ inhibition is used in animal testing and initial data, disclosed below, show P₅₀ prepulse inhibition for ICV clozapine at doses of 1/100^{th} to 1/500^{th} of oral dosing. Clozapine, and its dimethyl metabolite, have had CSF levels and serum levels studied clinically in chronically treated patients which revealed CSF/serum concentrations on the order of 1:15 suggesting that lower total doses can be administered ICV than through an oral route (Nordin et al. 1995).

The deficit in sensory inhibition can be quantified using the paired stimulus paradigm in which 2 identical stimuli are delivered 0.5 seconds apart and the electrophysiological response to each is recorded. In normal individuals, the response to the second, or test, stimulus, occurring 50 msec after stimulus onset, is reduced compared to the response to the first, or conditioning stimulus. However, schizophrenia patients have similar magnitude responses to both stimuli. The “TC ratio” is calculated by dividing the test amplitude by the conditioning amplitude. When the test amplitude is reduced, compared to the conditioning amplitude, the resultant TC ratio is less than 1. In normal individuals, the TC ratio is generally less than 0.4 while schizophrenia patients commonly have TC ratios above 0.5 and often approaching or exceeding 1.0.

In the present study, briefly, 5 baseline records were obtained in response to the paired auditory stimuli, prior to drug administration. Then, either 0.5 or 1 μg of clozapine were slowly (over about 30 sec) administered through a 26 gauge needle inserted into the anterior lateral ventricle, contralateral to the recording electrode. Recordings were obtained at 5 minute intervals for 90 minutes post injection. Data analyzed included the amplitude of the response to the first stimulus (conditioning amplitude), amplitude of the response to the second stimulus (test amplitude) and the TC ratio (test amplitude/conditioning amplitude). This final parameter gives a measure of the level of inhibition in the circuit initiated by the conditioning stimulus. TC ratios greater than 1 indicate that there has been no inhibition of the response to the second stimulus, while TC ratios <0.50 indicate normal sensory inhibition. DBA/2 mice routinely have TC ratios of ≧0.8.

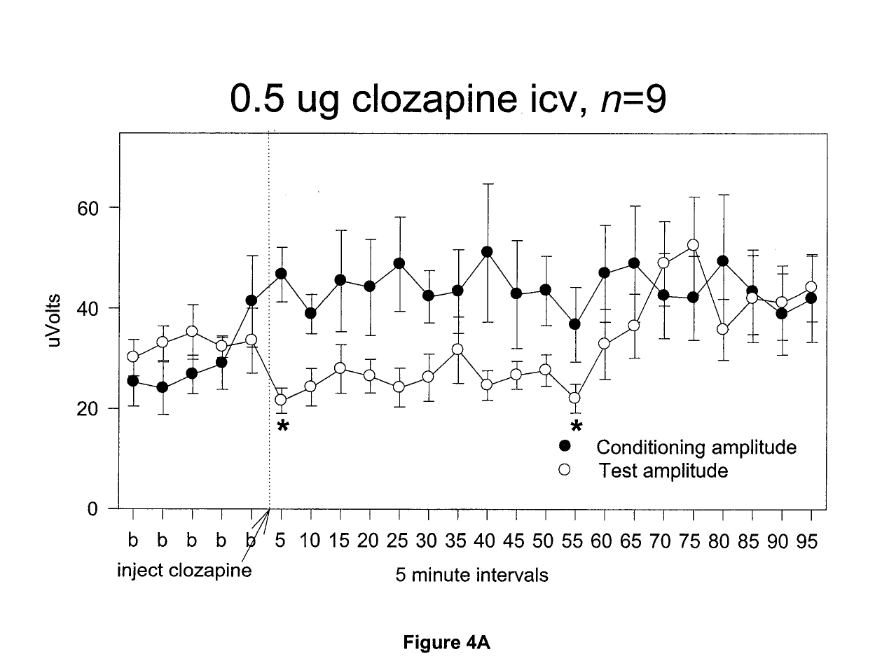

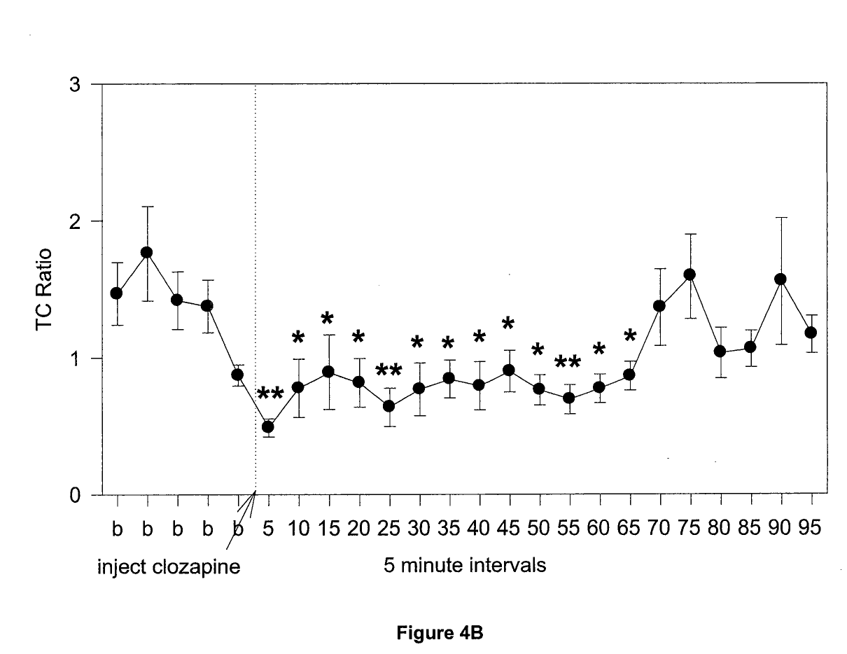

Repeated measures analysis of variance (ANOVA) for the 0.5 μg dose showed significant changes in TC ratio over time (F_{(23,184)}=3.07, p<0.001). Fisher's LSD a posteriori analysis showed that TC ratios were reduced beginning right after injection and remained reduced for over an hour before moving back towards pre-clozapine baseline levels. With reference to FIGS. 4A and 4B, centrally administered clozapine resulted in significantly reduced TC ratios compared to baseline which were produced by decreases in test amplitude and increases in conditioning amplitude, though the latter did not reach statistical significance. Data are mean±SEM; *p<0.05.

Analysis of condition and test amplitudes revealed that while there were no significant changes in conditioning amplitude (F_{(23,184)}=1.48, p=0.083) there were significant decreases in test amplitude (F_{(23,184)}=2.80, p<0.001). Fisher's LSD found 2 time points significantly reduced for test amplitude, but a general trend towards lower amplitudes compared to pre-drug baseline (FIG. 4B). Examination of FIG. 4B shows that, even though there was no significant change in conditioning amplitude, there was a trend towards increase in response amplitude.

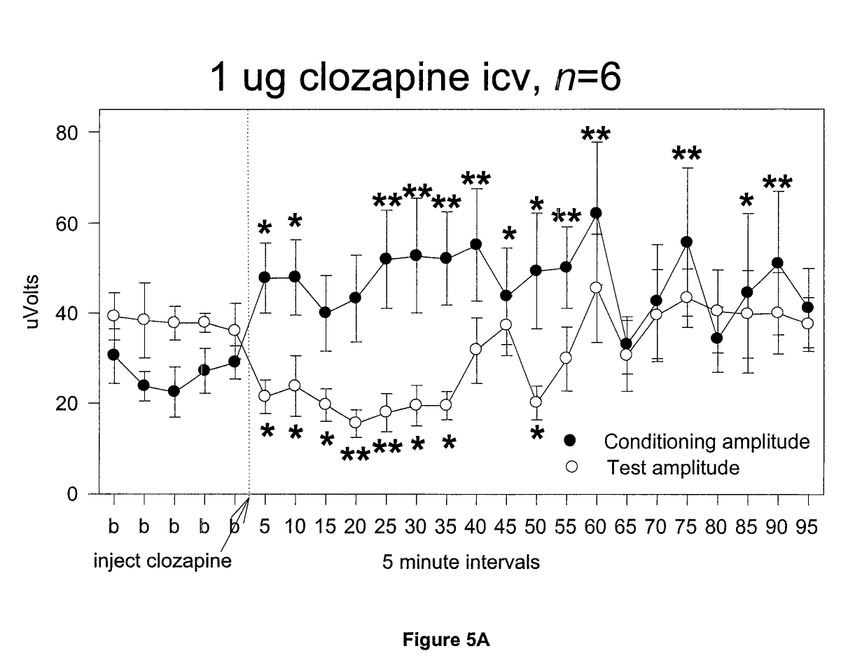

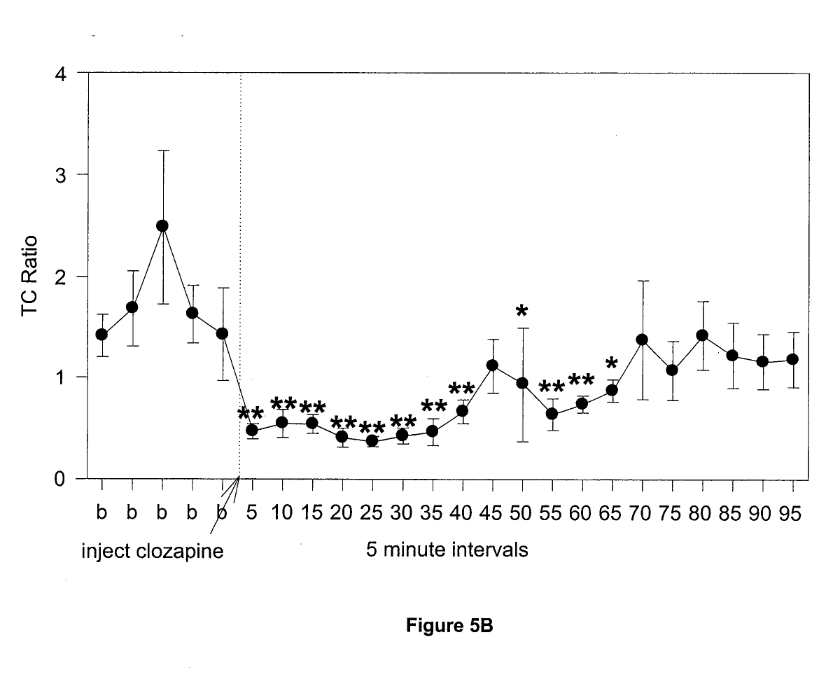

Similar analyses for the 1.0 μg dose of clozapine again showed significant changes in TC ratio (F_{(23,115)}=3.08, p<0.001) with significantly reduced TC ratios at similar time points to the 0.5 μg dose (Figure E). Conditioning and test amplitudes were also significant (F_{(23,115)}=2.50, p<0.001; F_{(23,115)}=2.58, p=0.001, respectively). Fisher's LSD showed significantly increased conditioning amplitudes throughout most of the recording session and significantly reduced test amplitudes for the first 35 minutes post injection. With reference to FIGS. 5A and 5B, similar to the 0.5 μg dose, there were significant decreases in TC ratio which were produced by decreases in test amplitude and increases in conditioning amplitude, both of which reached significance at this dose. Data are mean±SEM; *p<0.05; **p<0.01, compared to baseline.

These data are in concert with the effects of systemically administered clozapine in the same mouse model (Simosky et al 2002) but using more than a 1000-fold lower dose. In that study, it was found that significantly reduced TC ratios produced by significantly increased conditioning amplitudes and significantly reduced test amplitudes at a dose of 1 mg/kg. These changes in amplitude response to the auditory stimuli were produced not by direct action of clozapine at cholinergic receptors but indirectly by increased release of acetylcholine.

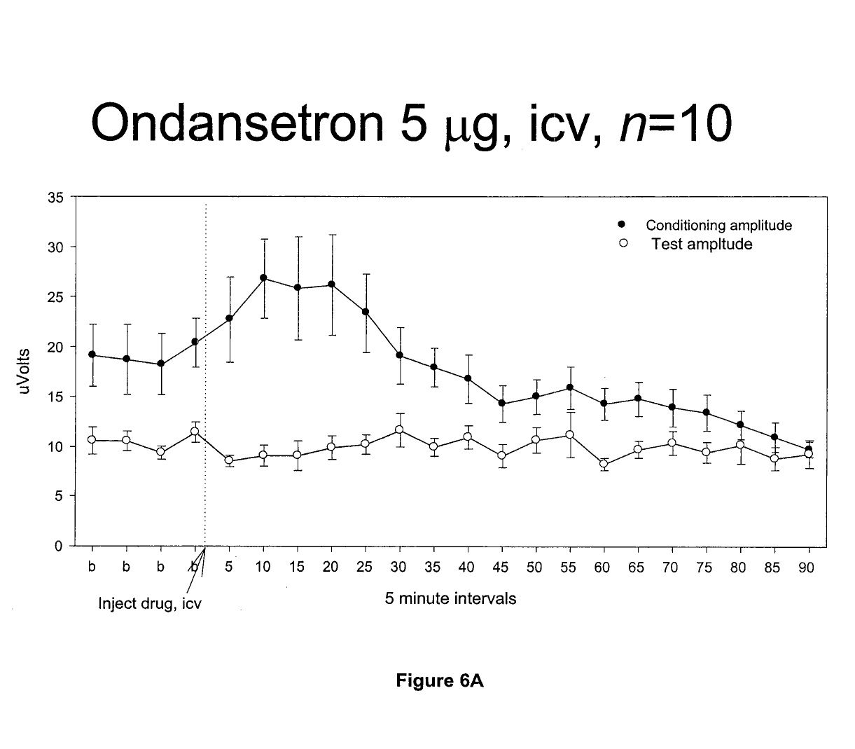

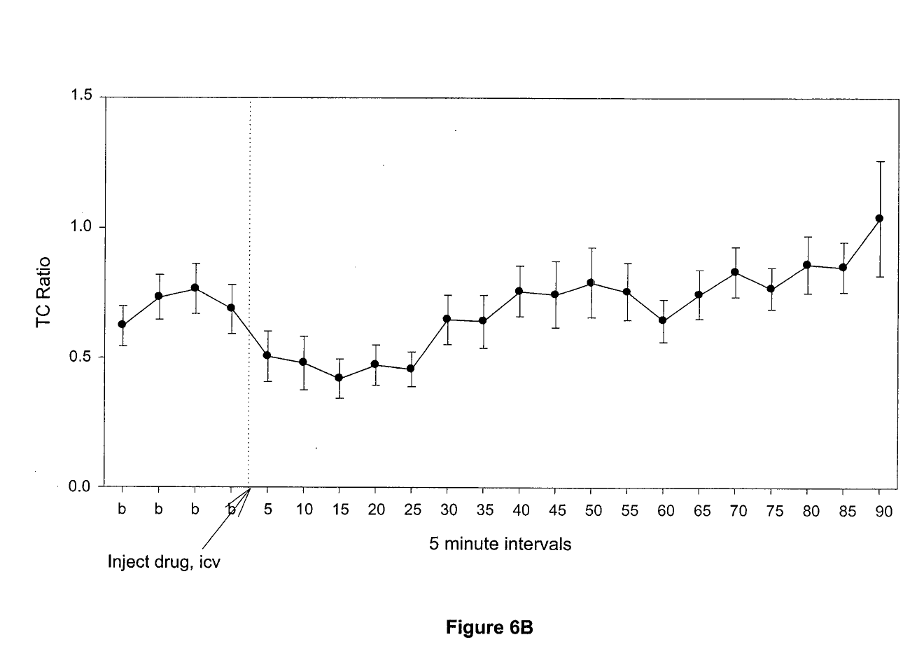

Again, similar analyzes for a 5 μg dose of ondansetron showed significant changes in TC ratio with significantly reduced TC ratios at similar time points to the dose administration (FIGS. 6A and 6B).

These data demonstrate the feasibility of administering active agents centrally to produce improvements in a rodent model of deficient sensory processing in schizophrenia patients at significantly lower dosages. Improvements in sensory inhibition in patients have been correlated with improvements in other symptoms of schizophrenia, suggesting that centrally administered agents in patients may improve other schizophrenia symptoms as well but using significantly smaller doses, thus avoiding side effect problems.

B: ICV Epilepsy Drug Efficacy and Epilepsy mediation Solubility Formulation

The following active agents therapeutically effective in the treatment of epilepsy were formulated in compositions of the present invention and ICV administered to rats in the pentylenetetrazole (PTZ) seizure induction model (Kupferberg 2001). The test agents reduced seizure frequency when administered with the PTZ. The data demonstrate the feasibility of administering the active agents centrally to produce improvements in seizure frequency at significantly reduced dosages, as compared to non-central treatment protocols.

The below formulations were observed to have no change in UV absorbance or detectable precipitation for at least 4 months at 37 degrees.

| | | | | | two-tailed T-test | Active | HP-beta- |
|---|---|---|---|---|---|---|---|
| | Drug | N | Mean | % Change | (p-value) | Conc. | cyclodextrin |
| Felbamate | A-V | 5 | 1.3 | 60 | 0.025 | 17.3 mM | 6.79% |
| | A-1 | 5 | 1.9 | | | | |
| Adenosine | B/E-V | 5 | 0.9 | 90 | 0.016 | 0.25 mM | saline |
| | B-1 | 4 | 1.8 | | | | |
| Lamictal | C-V | 7 | 0.9 | 60 | 0.0004 | 0.5 mM | 19.60% |
| | C-1 | 8 | 1.5 | | | | |
| Bumex | D-V | 5 | 1.3 | 90 | 0.0001 | 0.05 mM | 1.64% |
| | D-1 | 3 | 2.2 | | | | |
| Valproate | B/E-V | 5 | 0.9 | 60 | 0.020 | 50 mM | saline |
| | E-1 | 3 | 1.5 | | | | |
| Tegretol | F-V | 5 | 1.2 | 60 | 0.0002 | 1.88 mM | 2.28% |
| | F-1 | 4 | 1.8 | | | | |

Felbamate 17.3 mM 6.79% HP-Beta-Cyclodextrin
Adenosine 0.25 mM Saline
Lamictal 0.5 mM 19.6% HP-Beta-Cyclodextrin
Bumex 0.05 mM 1.64% HP-Beta-Cyclodextrin
Valproate 50 mM Saline
Tegretol 1.88 mM 2.28% HP-Beta-Cyclodextrin

### C: ICVAdministration of Anti-Depressants—Anxiety Animal Models

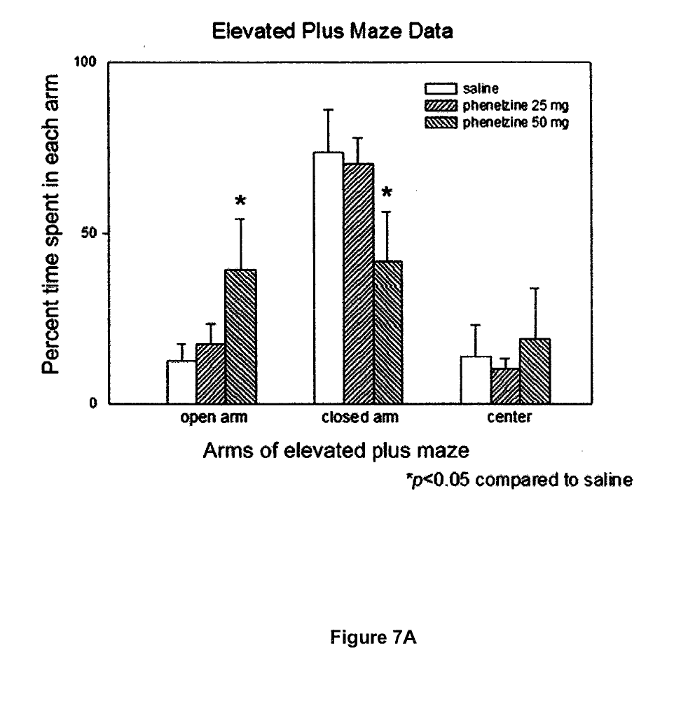

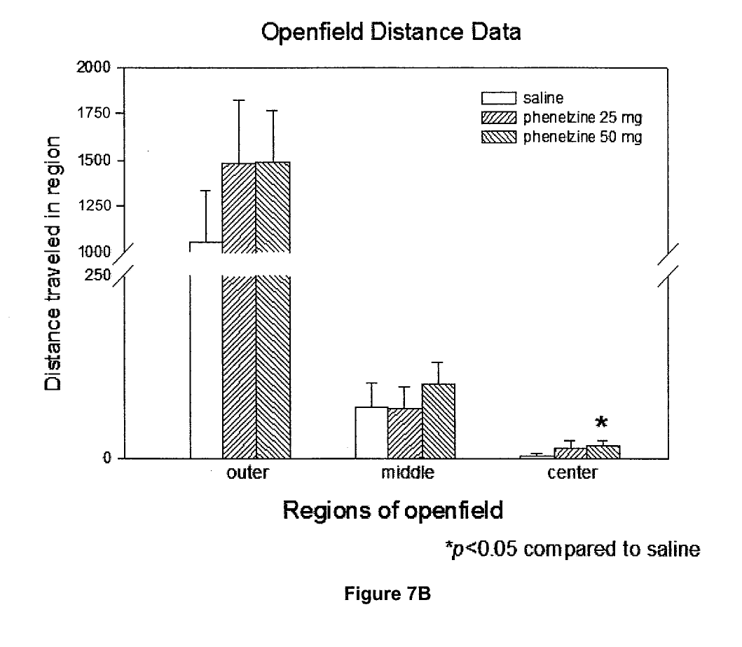

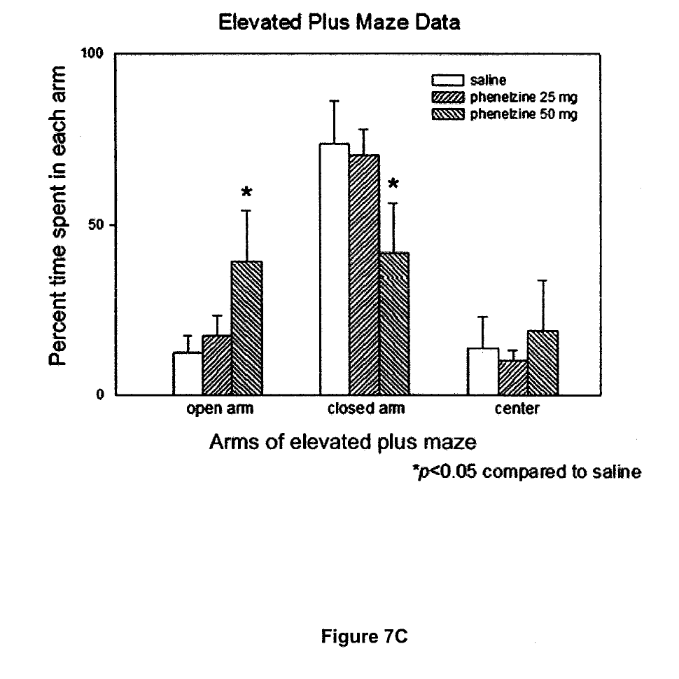

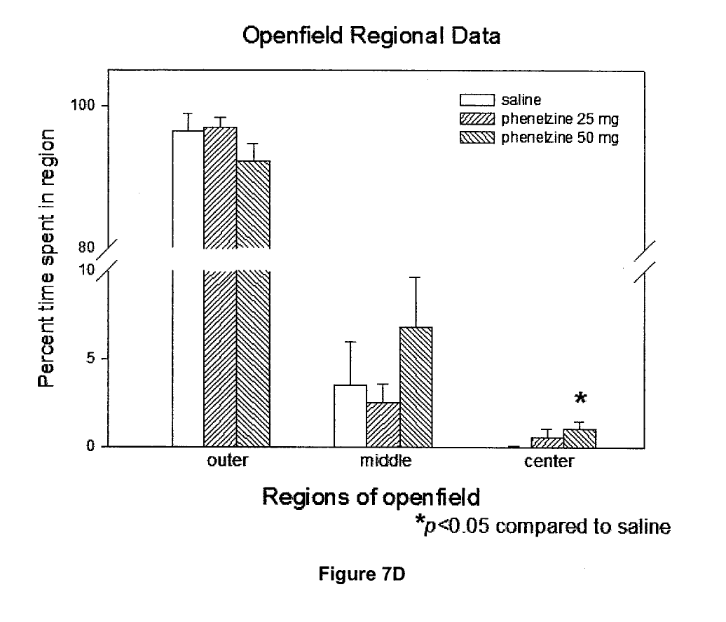

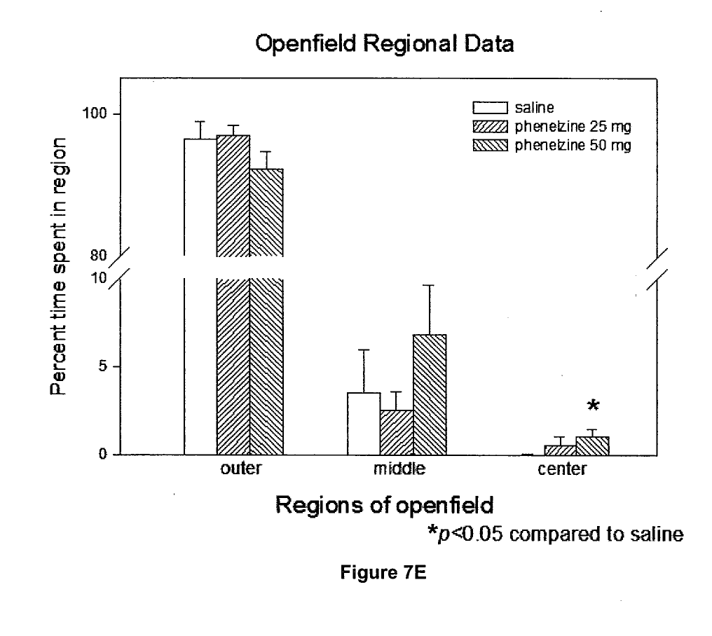

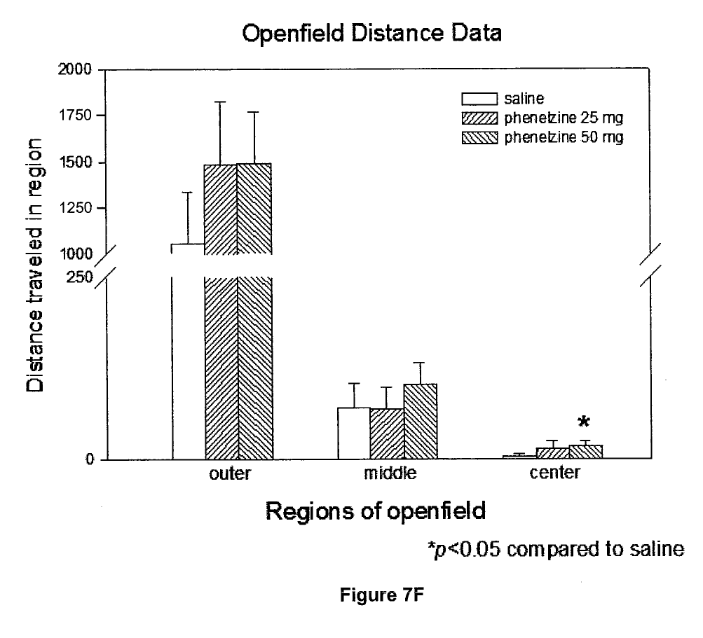

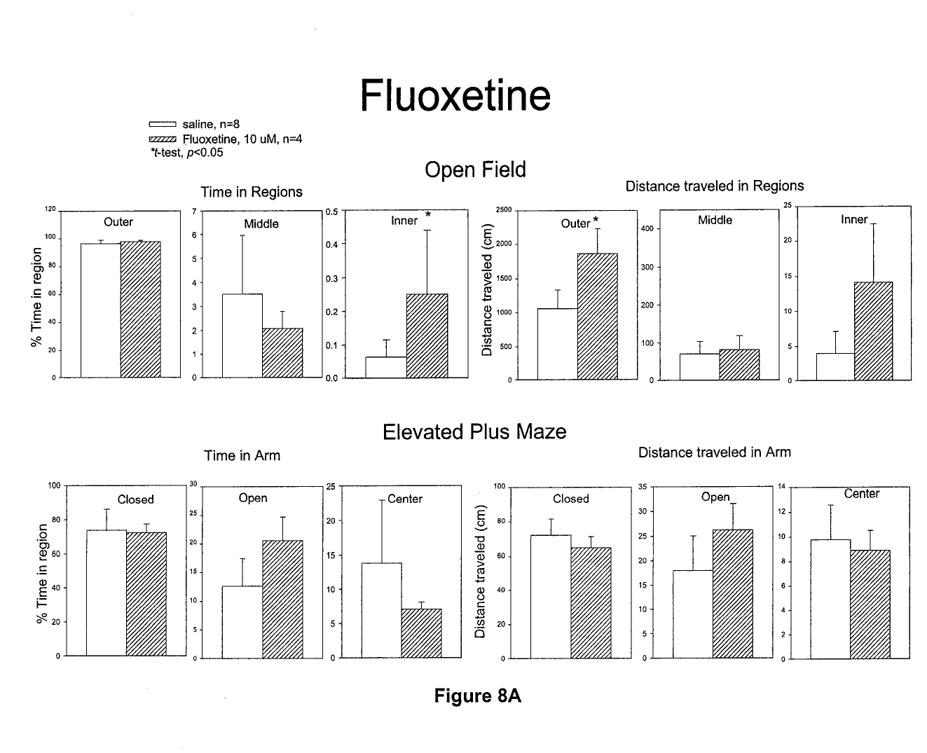

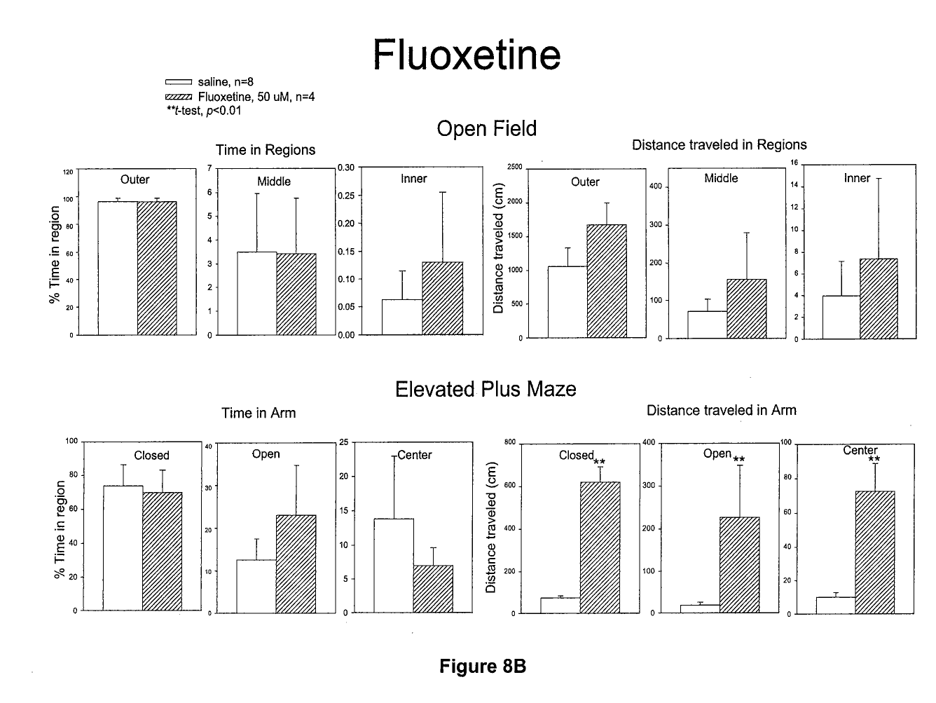

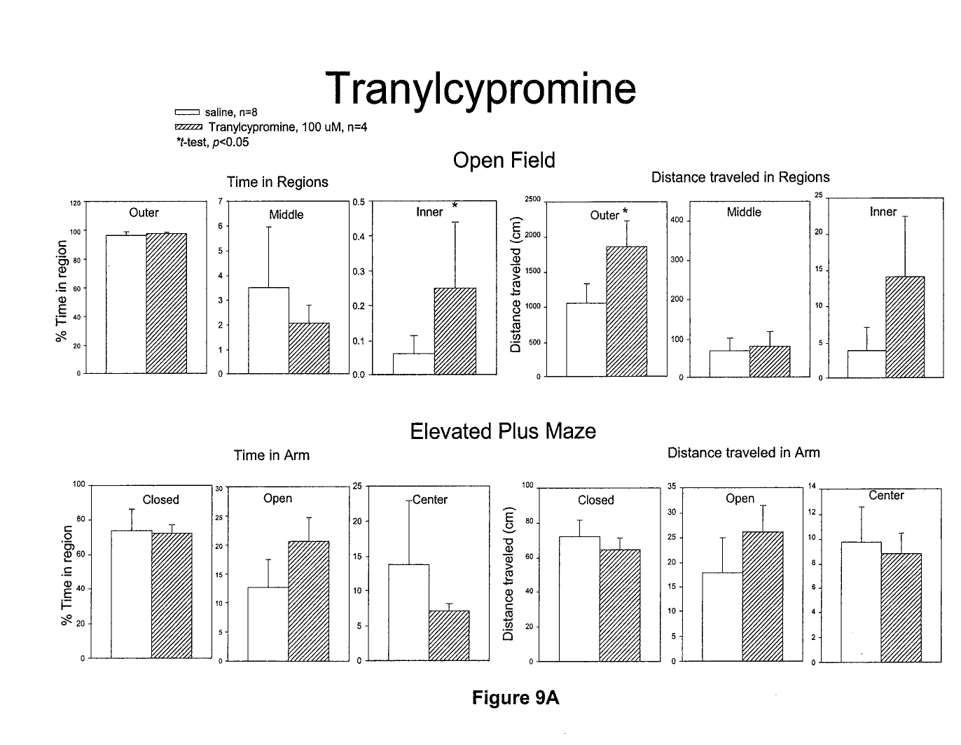

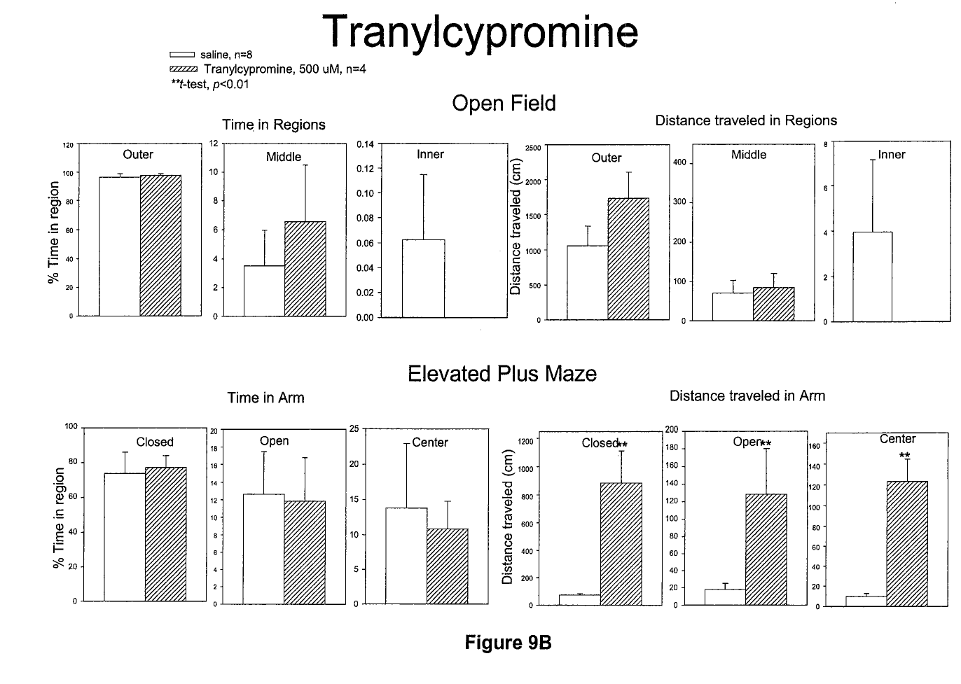

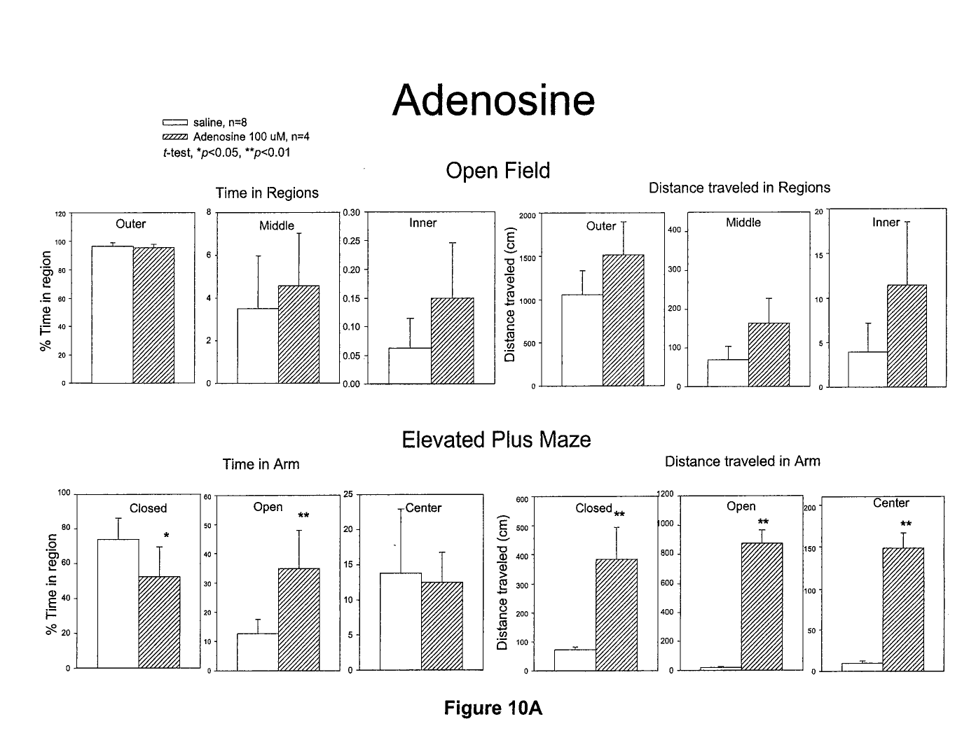

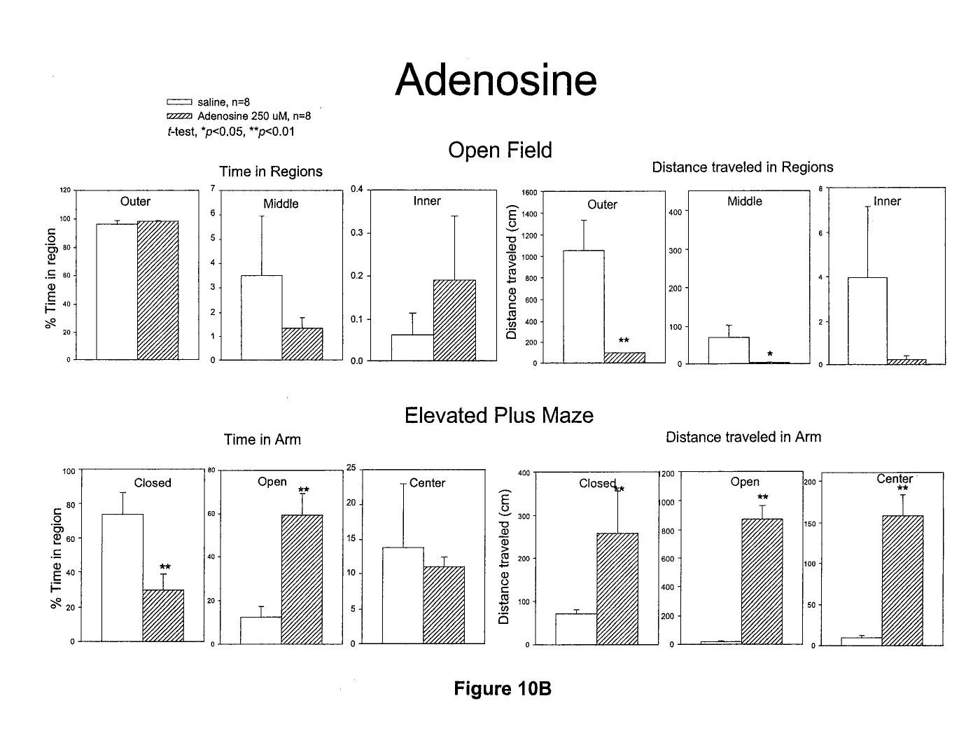

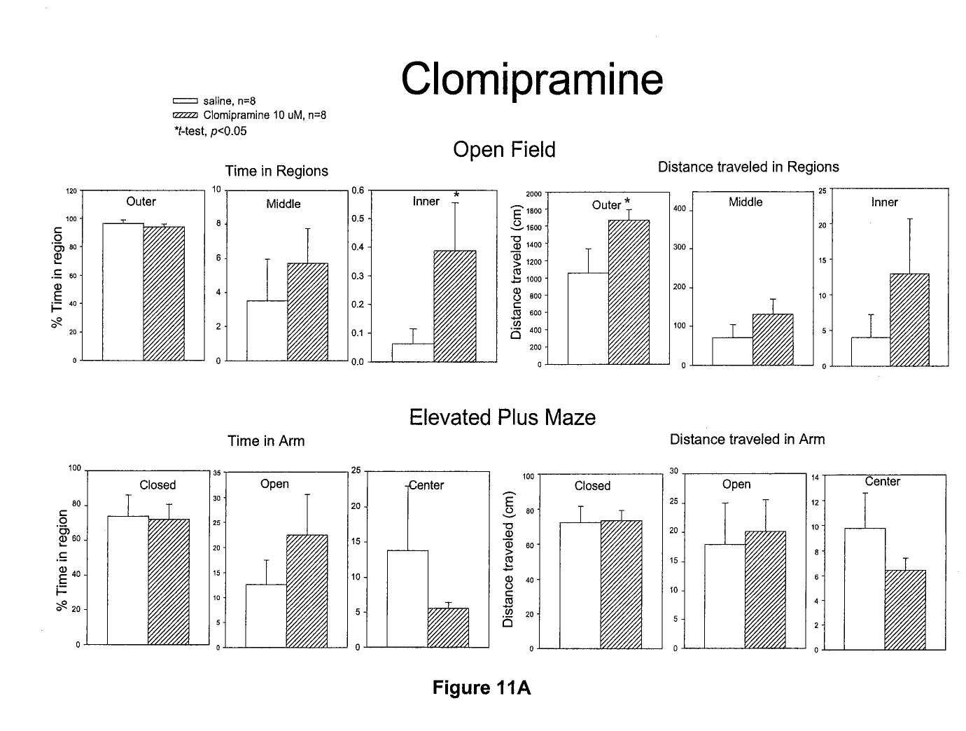

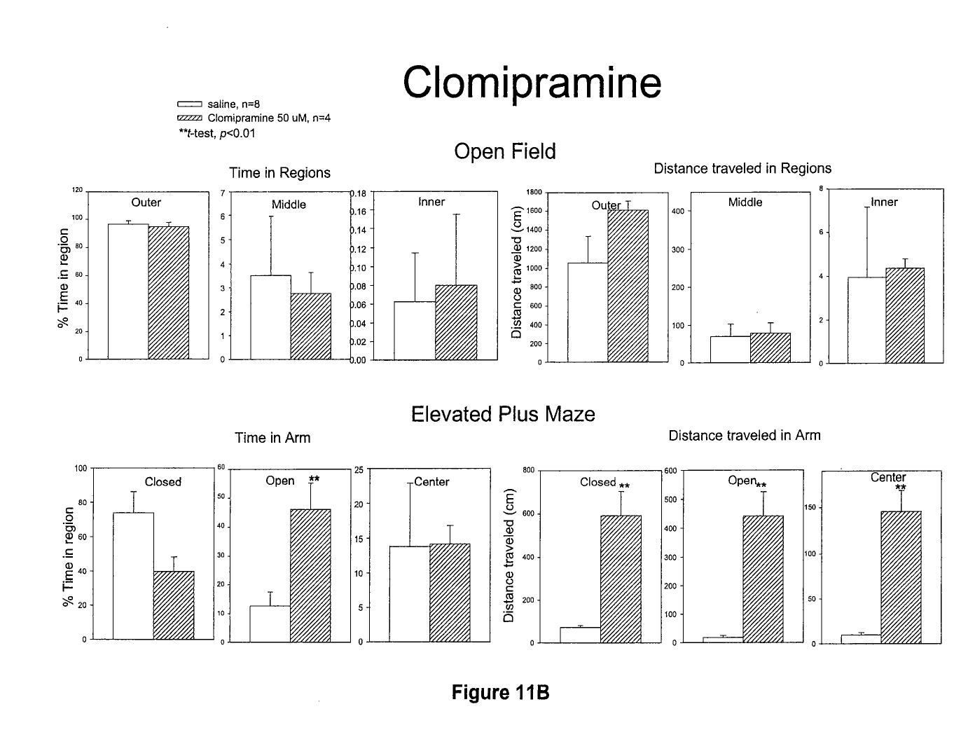

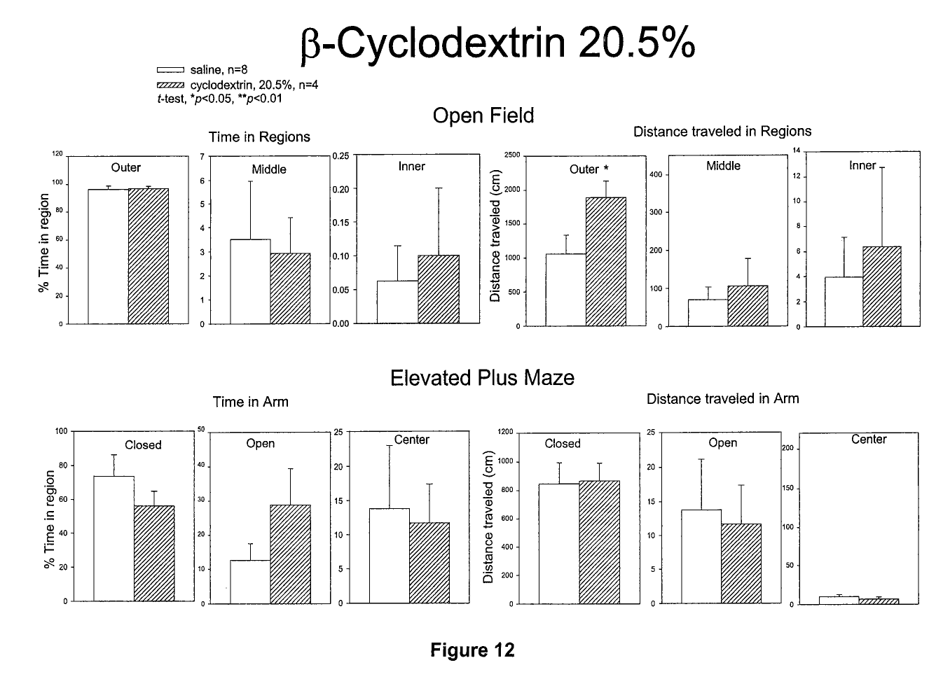

Various antidepressants were injected via ICV, and animals monitored in standard elevated plus maze and open field conflicts test. The data demonstrate the efficacy of various antidepressant following ICV administration (e.g., phenelzine, fluoxetine, tranylcypromine, adenosine, clomipramine, and cyclodextrin and saline as controls). (See FIGS. 7A-F, 8A-8B, 9A-9B, 10A-10B, 11A-11B, and 12)

The elevated plus and open field mazes can demonstrate decreased anxiety through increased activity in regions of the maze thought to be more prone to anxiety production (i.e. the open arms of the elevated plus and the central regions of the openfield maze) (Mechiel Korte and De Boer 2003; Crawley 1985). The swim tank can demonstrate decreased depression by increased struggle time to escape the water (Russig et al 2003).

### Example 3

### Chronic Central Administration and Brain Distribution of Active Agent

To determine steady state brain penetration and distribution of the active agent, a group of Sprague Dawley rats are implanted with a ventricular cannula attached to an osmotic minipump containing tritiated active agent in the excipient. After 14 days, the rats are sacrificed under anesthesia, the brain dissected out, frozen and sectioned. Sections are apposed to tritium sensitive film; the film exposed, developed and levels of binding assessed. Coefficients of penetration are determined for each region/formulation and compared to the active agent in saline. Liver, kidney, heart, skeletal muscle and/or eye tissue may also be analyzed if desired.

### A: Central Administration of Clozapine in Schizophrenia Model

Sprague Dawley rats, which have been prenatally stressed to produce deficient sensory inhibition at adulthood similar to that seen in both schizophrenia patients and the DBA/2 mice used above (Koenig et al 2003), are implanted with chronic recording electrodes (Steven et al 1991; 1993; 1995) and a cannula placed into the anterior ventricle with a catheter tube attached. A second cannula, closed with a stylette is placed in the other anterior ventricle. After 1 week recovery from surgery, at least 10 baseline recording sessions are performed in which 30 pairs of identical auditory click stimuli are presented and the evoked potentials are recorded and averaged. This establishes the baseline parameters for sensory inhibition in the rats.

Formulations described above are administered into the ventricles using an osmotic minipump to deliver 0.5 μl/hr for 14 days. The rats have a chronic recording electrode implant that allows repeated awake recording over several days and a ventricular cannula to permit withdrawal of CSF. Sensory inhibition is recorded on alternate days for the 14 days of the pump duration. At the end of each recording session, blood and CSF are sampled under light anesthesia to assess levels of the active agent. Brain penetration and distribution are assessed using tritiated active agent/excipient complex in the osmotic minipump in a separate group of animals. For comparison purposes, tritiated active agent is injected, IP, to allow us to directly compare tissue accumulation of radiolabeled drug between the injection modalities. A rat model of deficient sensory inhibition is used which allows us to sample both fluids repeatedly over several days.

To directly compare IP versus ICV administration of tritiated clozapine for brain penetration and tissue accumulation, 4 groups of rats are injected with the dose of clozapine which improved sensory inhibition in a previously published study (10 mg/kg ip, Simosky et al 2003). The rats are sacrificed at 6 hours post injection, a time roughly equal to 4 times the half life of clozapine in rats (Baldarassinni et al 1993) at which time steady state with plasma and brain/CSF should be achieved. The brain is dissected out, frozen and processed for autoradiography. Blood, CSF are collected and kidney, liver, skeletal muscle and eye taken.

Then an osmotic minipump containing the clozapine formulation is attached to a catheter connected to the cannula in the ventricle and placed under the skin of the upper back. Two days later, alternate day recording of sensory inhibition begins and continues for the full 14 days of the pump. At the end of each recording session, rats are lightly anesthetized with isoflurane and a 0.1 ml blood sample drawn from the femoral vein and 5 μl of CSF drawn from the other ventricular cannula for determination of the clozapine levels and the brain/plasma ratio. At the end of the last recording session, the rat is anesthetized and decapitated, the brain removed, placement of the cannulas in both ventricle verified, and the brain regionally dissected (hippocampus, striatum, anterior cortex, thalamus). The levels of clozapine in each region are determined. Data are analyzed by analysis of variance and appropriate a posteriori analyses performed wherever significant differences are found (p<0.05).

Chronically ICV delivered clozapine formulations attain a steady state level of clozapine in both the CSF and the plasma and the plasma levels are extremely low or not detectable, coincident with improvement in sensory inhibition, showing that we can achieve improvement in sensory inhibition deficits while maintaining plasma levels of clozapine far below that which induces agranulocytosis.

### Example 4

### CNS Toxicology

These studies demonstrate minimal or no CNS pathology and low systemic toxicity in rats administered ICV clozapine formulations for up to 14 days. At necropsy, blood is collected via cardiac puncture and placed in Na-EDTA anticoagulant or serum-separator tubes (SST). Anticoagulant blood is used to generate complete blood counts (CBCs). SST blood is spun down and serum collected to generate biochemical profiles CSF is collected via a cisterna magna puncture.

Tissues collected at necropsy for histopathology analysis include brain, skeletal muscle, eye, liver and kidney, and are preserved in 10% neutral-buffered formalin (5:1 formalin to tissue) for a minimum of 48 hours prior to processing. Tissues are processed for routine light microscopic analysis. Briefly, tissues are dehydrated, imbedded in wax, cut into 8 μm sections and mounted on slides, re-hydrated, and hematoxylin/eosin stained (H&E).

There are no statistically significant differences between the blood and tissue parameters examined between and treated and control animals. Biochemical and CBC values are pooled by treatment group and means compared to sham control group values using paired t-tests. Histopathology samples are assigned a point value based on the degree of necrosis, inflammatory cell infiltrate, and fibrosis. Scores for each are summed by group and compared to sham control tissues.

The following studies demonstrate that chronic ICV administration of clozapine results in significantly less accumulation of drug in peripheral tissues and organs than intraperitoneal (IP) clozapine administration. Life-threatening effects of oral clozapine administration, such as myocarditis and agranulocytosis, are attributable to the elevated systemic drug levels necessary to achieve therapeutic concentrations in the CNS. ICV administration drastically reduces the dose needed, and thus the toxic side effects. This experiment compares the tissue distribution of clozapine in ICV versus IP (systemic) drug administration.

Tissues from euthanized animals are collected and drug levels quantitated as follows: Tissues are recovered, place in an Eppendorf tube and weighed. Tissue solubilizer (Biolute-S, Serva Electrophoresis) is added and the mixture allowed digesting for a minimum of twelve hours on a rocking platform. Digests are then mixed with scintillation fluid (Scintisafe, Fisher Scientific, 50:50 v/v) and counts quantitated utilizing a Beckman model LS 6500 scintillation counter.

Counts are normalized to initial tissue weights and drug distribution comparisons made between ICV and IP delivery routes. ICV delivery results in statistically significant reductions in all peripheral tissues when compared to systemic drug delivery.

### Example 5

### Methods of Treating Schizophrenia and Psychotic Disorders

Olanzapine, Geodon, Aripiprazole, and Quetiapine have been used for systemic treatment of schizophrenia and psychotic disorders. Problems with medication side effects, adherence and tolerance have limited its usefulness. Central administration of the active agents, as discussed in the Examples above for clozapine administration to schizophrenia patients, substantially reduces systemic effects by decreasing circulating blood levels of the active agent, while providing efficacious therapeutic alleviation of psychotic symptoms.

A 5 mg/ml solution of the active agent is solubilized in aqueous solution using beta-hydroxypropyl cyclodextrin, made isotonic with NaCl, and the pH is maintained at 7.4 with 10 mM sodium phosphate. An antioxidant comprised of modified vitamin E compounds, (e.g., Trolox or PEG-Tocopherol succinate) at between 50 micrograms/mL to 1 mg/mL is then optionally added to the mixture. The stabilized solution is inserted into a fluid reservoir attached to a Medtronic Synchromed-II intrathecal delivery system. The stabilized formulation is intracerebroventricularly or cistema magna injected into patients diagnosed with psychotic disorders.

The patient population is selected from individuals for whom standard schizophrenic therapy has been ineffective at alleviating symptoms. Injection is continuous, using a computerized pump to provide a delivery rate of 0.01 to 0.1 mg of the active agent per hour, depending on the severity of symptoms. CSF concentration is periodically monitored and the delivery rate is adjusted accordingly to provide a steady-state concentration of 1 to 5 micrograms per milliliter of cerebrospinal fluid. After 1 week of treatment, schizophrenic symptoms are alleviated.

### Example 6

### Methods of Treating Epilepsy

Felbatol, Bumetanide, Carbamazepine, and Phenyloin have been used for systemic treatment of epilepsy. Problems with medication side effects have limited its usefulness. Central administration of the active agents, as discussed in the Examples above for clozapine administration to schizophrenia patients, substantially reduces systemic effects by decreasing circulating blood levels of the active agent, while providing efficacious therapeutic alleviation of seizures.

A 5 mg/ml solution of active agent is stabilized and/or solubilized using optional beta-hydroxypropyl cyclodextrin, made isotonic with NaCl, and the pH is maintained at 7.4 with 10 mM sodium phosphate. An optional antioxidant of modified vitamin E compounds, (e.g., Trolox or PEG-Tocopherol succinate) at 50 micrograms/mL to 1 mg/mL is added to the mixture. The stabilized solution is inserted into a fluid reservoir attached to a Medtronic Synchromed-II intrathecal delivery system. The stabilized formulation is intracerebroventricularly or cistema magna injected into patients diagnosed with epilepsy disorders.

The patient population is selected from individuals for whom standard epilepsy therapy has been ineffective at alleviating symptoms. Injection is continuous, using a computerized pump to provide a delivery rate of 0.01 to 0.1 mg active agent per hour, depending on the severity of symptoms. CSF concentration is periodically monitored and the delivery rate is adjusted accordingly to provide a steady-state concentration of 1 to 5 micrograms per milliliter of cerebrospinal fluid. After 1 week of treatment, epileptic frequency is reduced.

### Example 7

### High Concentration Formulations for Use in a Delivery Apparatus for Central Administration

The technique for formulating the drug compositions was essentially that described for clozapine in Example 1. The formulations were obtained by titrating the drug with beta-cyclodextrin (HPBCD) when necessary to solubilize the drug, and then titrating the composition to neutral pH. The table below shows the drug concentrations obtained and the HPBCD concentrations in the formulations. In this case the volume in the delivery apparatus is approximately 15 ml or 40 ml.

| | | Human ICV | Highest conc. | HPBCD |
|---|---|---|---|---|
| Drug | MW | mg/ml | Made mg/ml | conc. |
| ▴ Felbamate | 238.24 | 32.94 | 12.3 | 20.40% |
| Adenosine | 267.24 | 0.534 | 3.34 | NONE |
| Lamictal | 256.09 | 1.023 | 6.4 | 19.60% |
| Bumex | 364.42 | 0.145 | 0.92 | 1.64% |
| Valproate | 166.19 | 57.62 | 58.24 | NONE |
| Tegretol | 236.27 | 3.55 | 3.67 | 9.90% |
| Clomipramine | 351.31 | 147.85 | 148 | 20% |
| ▴ Phenelzine | 234.17 | 719.28 | 308.3 | NONE |
| ▴ Fluoxetine | 345.79 | 295.7 | 147.9 | 30% |
| Tranylcypromine | 169.65 | 12.75 | 12.75 | NONE |
| Clozapine | 316.82 | 15.98 | 17 | 27.20% |

▴: Considering the pump volume can be increased from 15 ml to 40, the concentration can be at least 2.7 times lower than the Human ICV concentration.

### Example 8

### Methods to Determine the Highest Non-Toxic and Efficacious Concentration of a Drug

Several animal models as described below were used to determine the efficacy of formulations containing different concentrations of the drug. The animals were also tested for toxic effects as described in the table, below. The table also shows the highest non-toxic concentration of the drug that also exhibited efficacy

### A. Methods for Determining Efficacy

1. Clozapine: Improvement in Rodent Sensory Inhibition Deficits which Model Abnormal Sensory Inhibition Observed in Schizophrenia.

DBA/2 mice are implanted with a chronic indwelling cannula aimed at one anterior lateral ventricle (Alzet Brain Infusion system) which is connected to an osmotic minipump delivering 0.25 μl/hr for 7 days. On the 7^{th} day, the mouse is anesthetized, placed in a stereotaxic instrument, a burr hole opened in the skull over the contralateral dorsal hippocampus and a recording electrode lowered to the CA3 region of the hippocampus. Evoked EEG responses to closely paired (0.5 msec apart) identical tones are recorded. In normal sensory inhibition, the response to the second tone is diminished compared to the response to the first tone. DBA/2 mice show similar responses to both tones as do schizophrenia patients. Chronic administration of clozapine to DBA/2 mice produced significant decreases in the response to the second tone, producing normal sensory processing.

### 2. Adenosine, Felbamate, Tegretol, Lamictal, Bumex, Valproate: Improvement in Acute Seizure Induction in the Rodent Model of Epilepsy-Like Seizures.

Rats are anesthetized, placed in a stereotaxic instrument and a burr hole opened over one anterior lateral ventricle. A cannula is lowered to the ventricle and 5 μl of solution containing the drug is injected into the ventricle over 10 minutes. The incision is then closed and the animal allowed to regain consciousness and remain conscious for 10 minutes. After 10 minutes has elapsed, pentylenetetrazole (PTZ, 10 mg/ml in saline) is injected through a tail-vein catheter at a constant rate of infusion (1 m/min, Razel syringe pump) until a motor seizure is induced. PTZ seizures are identified as myoclonic jerking followed by clonus and tonus. The time from the initial application of PTZ to the first clonic seizure (i.e., forelimb clonus) is measured and defined as “latency to seiqure onset”. The total amount of PTZ infused is calculated and then divided by each rat;s body weight to obtain the dose (mg/kg) of PTZ. The dose required to attain status epilepticus is compared to the PTZ dose without antiseizure medication.

### 3. Phenzine, Tranylcypromine, Lamictal: Reduction in Quiescent Time in Prosolt Forced Swim Test of Behavioral Despair (Depression).

A 20 cm diameter, 56 cm tall acrylic cylinder is filled with about 45 cm of water (35° C.) and the rat placed in the water for 5 minutes and behavior monitored. The amount of time spent quiescent (non-struggling) is compared between animals receiving the drug and those receiving saline controls. A reduction in quiescent time is thought to reflect a reduction in behavioral despair.

### 4. Fluoxetine, Clomipramine:

a. Increased Time and Distance Traveled in Open Arm of Elevated Plus Maze (Anxiolytic Action).

Rats are placed in the center of a pluz-shaped maze. The arms are 13 cm wide; two arms were open and two are enclosed by 32 cm high walls. The maze is elevated off the flor by 51 cm. The percent of time and distance traveled in each type of arm over the 4 minute test period is calculated by computer. Increased time and distance traveled in the open arms is thought to reflect anxiolytic activity.

b. Increased Time and Distance Traveled in the Center Area of an Openfield Maze (Anxiolytic Action).

Rats are place against 1 side of a ‘104 cm square box with 32 cm sides. The activity of the rat is monitored by computer over a 4 minute testing period. A computer divides the openfied space into 25 equal squares arranged into an outer :ring” of 16 squares, a middle region of 8 squares and a central square. The percent of time and distance traveled in the center square by rats receiving the drug are calculated and compared to rats receiving a saline control. Increased time and distance traveled in the center square is thought to reflect anxiolytic activity.

### 5. All Drugs: Ataxia Scale.

All animals are assessed for ataxia, which is a measure of altered gate and ability to change body position. They are also placed on their acks and time to “righting” measured. No animal showed altered gate and no animal showed delay in righting.

B. Toxicology

The table below shows the drugs tested and the methods of assessing the toxicological effects of some of the drugs that were tested for efficacy. The table also shows the highest concentration of the drug that exhibited efficacy in the test described under section A and that was non-toxic.

| | | Highest non-toxic |
|---|---|---|
| Drug | Method of assessment | concentration |
| Clozapine | In vitro primary cortical cell | 10 ug/ml |
| | culture | |
| | Behavioral: openfield, elevated | 50 mg/ml |
| | plus, ataxia | |
| Fluoxetine | Tissue pathology following | 0.0172 mg/ml |
| | minimum 28 day continuous | |
| | perfusion | |
| | Behavioral: openfield, elevated | 0.0172 mg/ml |
| | plus, ataxia | |
| Clomipramine | Tissue pathology following | 0.0176 mg/ml |
| | minimum 28 day continuous | |
| | perfusion | |
| | Behavioral: openfield, elevated | 0.0175 mg/ml |
| | plus, ataxia | |
| Tranylcypromine | Tissue pathology following | 0.0848 mg/ml |
| | minimum 28 day continuous | |
| | perfusion | |
| | Behavioral: openfield, elevated | 0.0848 mg/ml |
| | plus, ataxia | |
| Felbamate | Tissue pathology following | In process |
| | minimum 28 day continuous | |
| | perfusion | |
| | Behavioral: openfield, elevated | 0.056 mg/ml |
| | plus, ataxia | |
| Lamictal | Tissue pathology following | In process |
| | minimum 28 day continuous | |
| | perfusion | |
| | Behavioral: openfield, elevated | 0.384 mg/ml |
| | plus, ataxia | |
| Adenosine | Tissue pathology following | In process |
| | minimum 28 day continuous | |
| | perfusion | |
| | Behavioral: openfield, elevated | 0.0694 mg/ml |
| | plus, ataxia | |

### Example 9

### Animal Model Study for Treatment of Multiple Sclerosis

Drugs usually used for the treatment of Rheumatoid Arthritis are injected animals that are a known model for MS. The drugs are injected into the fluid around the brain.

### A. Preparation of the MS Model

Lewis rats, 6-8 weeks, obtained from Harlan, are immunized subcutaneously with 200 ml inoculum containing 25 mg Myelin Basic Protein (MBP 68-82 obtained from Sigma), 2 mg Mycobacterium tuberculosis (strain H37RA; Difco, Detroit, Mich.), 100 ml saline, and 100 ml Freund's incomplete adjuvant (DIFCO). Rats are weighed and evaluated daily or every other day in a blinded fashion for the presence of clinical signs. Clinical scores of EAE are graded according to the following criteria: 0, asymptomatic; 1, flaccid tail; 32, loss or righting reflex with or without partial hindlimb paralysis; 3, complete hindlimb paralysis; 4, moribund; 5, dead. After receiving MBP, the animals are monitored daily for these neurological signs and weighed every other day until death or sacrifice. All animals are sacrificed when moribund. At the time of death or sacrifice, the brains are harvested for histological examination.

### B. ICV Dose Response Curve-Treatment Arm

ICV (intracerebro-ventricular) anti-inflammatory drugs (prednisone, methotrexate, cyclosporine and indomethacin) are injected into the right ventricles of the animals after an Alzet pump is implanted subcutaneously. The Alzet pump is placed in the ventricle during an anaesthetic procedure. The pumps contain varying concentrations of the formulated drugs. The animals are awakened and allowed to recover from anaesthesia. ICV implantation occurs six days after MBP subcutaneous injection. ICV administration of the medications continues for eight days and all surviving animals are sacrificed.

Dose response curves are constructed using the four different drugs at four different doses. The starting effective dose is estimated from in vitro studies. Each treatment group contains 5 animals, as does the vehicle control group. The drugs are given ICV at a rate of 0.5 μl/hour, and the frequency of dosing is once and continuous. The duration of treatment effects is monitored between days 6 to 14 of the schedule. The doses for the drugs are in the following ranges: cyclosporine, 0.1 to 500 micromolar; methotrexate, 1 to 500 nanomolar; indomethacin 1 to 100 micromolar; and prednisone, 0.1 to 100 micromolar.

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the claims of the invention.

### LITERATURE CITED

Adler L E, Hoffer L J, Wiser A, and Freedman R. Transient normalization of a defect in auditory sensory processing in schizophrenics following cigarette smoking. Am. J. Psychiat., 150: 1856-1861, 1993.
Adler L E, Olincy A, Waldo M C, Harris J, Griffith j, Stevens K, Flach K, Nagamoto H, Bickford P, Leonard S and Freedman R. Schizohrenia, sensory gating, and nicotinic receptors. Schizophr Bull, 24:189-202, 1998.
Aravagiri, M, Marder, S R. Simultaneous determination of clozapine and its N-desmethyl and N-oxide metabolites in plasma by liquid chromatography/electrospray tandem mass spectrometry and its application to plasma level monitoring in schizophrenic patients. J Pharmaceut Biomed Analysis 26:301-311, 2001.
Allebeck P Schizophrenia: a life-shortening disease Schizophr Bull. 1989; 15(1):81-9. Alvir J M, Lieberman J A, Safferman A Z, Schwimmer J L, Schaaf J A. Clozapine-induced agranulocytosis. Incidence and risk factors in the United States. N Engl J Med. 1993 Jul. 15; 329(3): 162-7.
Anderson V C, Burchiel K: A prospective study of long-term intrathecal morphine in the management of chronic nonmalignant pain. Neurosurgery. 1999 February; 44(2):289-300; discussion 300-1.
Atkin K, Kendall F, Gould D, Freeman H, Liberman J, O'Sullivan D. Neutropenia and agranulocytosis in patients receiving clozapine in the UK and Ireland. Br J Psychiatry. 1996 October; 169(4):483-8.
Azorin J M, Spiegel R, Remington G, Vanelle J M, Pere J J, Giguere M, Bourdeix I. A double-blind comparative study of clozapine and risperidone in the management of severe chronic schizophrenia. Am J Psychiatry. 2001 August; 158(8):1305-13.
Blaney S M, McCully C L, Bacher J D, Murphy R F, Poplack D G. Methotrexate distribution within the subarachnoid space after intraventricular and intravenous administration Cancer Chemother Pharmacol. 2000; 45(3):259-64.
Bartels S J. Improving system of care for older adults with mental illness in the United States. Findings and recommendations for the President's New Freedom Commission on Mental Health. Am J Geriatr Psychiatry. 2003 September-October; 1 (5):486-97.
Blasberg R G, Patlak C S, Shapiro W R. Distribution of methotrexate in the cerebrospinal fluid and brain after intraventricular administration. Cancer Treat Rep. 1977 July; 61(4):633-41.
Blasberg R G, Patlak C, Fenstermacher M. Intrathecal chemotherapy: brain tissue profiles after ventriculocistemal perfusion. J Pharmacol Exp Ther. 1975 October; 195(1):73-83.
Blasberg R G. Methotrexate, cytosine arabinoside, and BCNU concentration in brain after ventriculocisternal perfusion. Cancer Treat Rep. 1977 July; 61(4):625-31.
Braff D. Information processing and attention dysfunction in schziophrenia. Schizophr Bull, 19:233-259, 1993.
Breese C R. Adams C. Logel J. Drebing C. Rollins Y. Barnhart M. Sullivan B. Demasters B K. Freedman R. Leonard S. Comparison of the regional expression of nicotinic acetylcholine receptor alpha7 mRNA and [125I]-alpha-bungarotoxin binding in human postmortem brain. J Comp Neurol. 387:385-98, 1997.
Breier A. Clozapine and noradrenergic function: support for a novel hypothesis for superior efficacy. J Clin Psychiatry. 1994 September; 55 Suppl B: 122-5.
Buchanan R W, Breier A, Kirkpatrick B, Ball P, Carpenter W T Jr. Positive and negative symptom response to clozapine in schizophrenic patients with and without the deficit syndrome. Am J Psychiatry. 1998 June; 155(6):751-60.
Caldwell C B, Gottesman H. Schizophrenia—a high-risk factor for suicide: clues to risk reduction. Suicide Life Threat Behav. 1992 Winter; 22(4):479-93.
Campbell A, Baldessarini R J, Teicher M H. Decreasing sensitivity to neuroleptic agents in developing rats; evidence for a pharmacodynamic factor. Psychopharmacology (Berl). 1988; 94(1):46-51.
Ciompi L. Learning from outcome studies. Toward a comprehensive biological-psychosocial understanding of schizophrenia. Schizophr Res. 1988 November-December; 1 (6):373-84. Cohen B M, Tsuneizumi T, Baldessarini R J, Campbell A, Babb S M. Differences between antipsychotic drugs in persistence of brain levels and behavioral effects. Psychopharmacology (Berl). 1992; 108(3):338-44.
Croft R J, Lee A, Bertolot J, and Fruzelier, M. Associations of P50 suppression and desensitization with perceptual and cognitive features of “unreality” in schizotypy. Biol Psychiatry. 50:441-6, 2001.
Collins J M. Pharmacokinetics of intraventricular administration. J Neurooncol. 1983; 1(4):283-91.
Crawley J N. Exploratory behavior models of anxiety in mice. Neuroscience & Biobehavioral Reviews. 9(1):37-44, 1985.
Erwin R J. Turetsky B I. Moberg P. Gur R C. Gur R E. P50 abnormalities in schizophrenia: relationship to clinical and neuropsychological indices of attention. Schizophrenia Research. 33:157-67, 1998.
Fleischhaker C, Schulz E, Tepper K, Martin M, Hennighausen K, Remschmidt H. Long-Term Course of Adolescent Schizophrenia Schizophrenia Bulletin 2005 31(3):769-780.
Freedman R, Hall M, Adler L E, and Leonard S. Evidence in postmortem brain tissue for decreased numbers of hippocampal nicotinic receptors in schizophrenia. Biol. Psychiatry. 38:22-33, 1995.
Ghersi-Egea J F, Finnegan W, Chen J L, Fenstermacher J D. Rapid distribution of intraventricularly administered sucrose into cerebrospinal fluid cisterns via subarachnoid velae in rat. Neuroscience. 1996 December; 75(4): 1271-88.
Goldberg T E, Torrey E F, Gold J M, Ragland J D, Bigelow L B, Weinberger D R. Learning and memory in monozygotic twins discordant for schizophrenia. Psychol Med. 1993 February; 23(1):71-85.
Grossman S A, Reinhard C S, Loats H L. The intracerebral penetration of intraventricularly administered methotrexate: a quantitative autoradiographic study. J Neurooncol. 1989 November; 7(4):319-28.
Harding C M, Brooks G W, Ashikaga T, Strauss J S, Breier A. The Vermont longitudinal study of persons with severe mental illness, II: Long-term outcome of subjects who retrospectively met DSM-III criteria for schizophrenia. Am J Psychiatry. 1987 June; 144(6):727-35.
Hasan N Y, Elkawy M A, Elzeany B E, Wagieh N E. Stability indicating methods for the determination of clozapine. J. Pharm. Biomed. Analysis 30:35-47 (2002).
Herz A, Albus K, Metys J, Schubert P, Teschemacher H. On the central sites for the antinociceptive action of morphine and fentanyl. Neuropharmacology. 1970 November; 9(6):539-51.
Iqbal M M, Rahman A, Husain Z, Mahmud S Z, Ryan W G, Feldman M. Clozapine: a clinical review of adverse effects and management. Ann Clin Psychiatry. 2003 March; 15(1):33-48.
Kaplan and Sadock's Comprehensive Textbook of Psychiatry Sadock and Sadock 2004.
Klonoff H, Fibiger C H, Hutton G H. Neuropsychological patterns in chronic schizophrenia. J New Ment Dis. 1970 April; 150(4):291-300.
Kupferberg H. Animal models used in the screening of antiepileptic drugs. Epilepsia. 42 Suppl 4:7-12, 2001
Lapchak P A, Araujo D M, Carswell S, Hefti F. Distribution of [125I]nerve growth factor in the rat brain following a single intraventricular injection: correlation with the topographical distribution of trkA messenger RNA-expressing cells. Neuroscience. 1993 May; 54(2):445-60.
Levin A Schizophrenia's Suicide Risk May Be Less Than Thought Psychiatric News Apr. 15, 2005 Volume 40 Number 8.
Levy, R. Implanted Drug Delivery Systems for Control of Chronic Pain. Chapter 19 of Neurosurgical Management of Pain. New York, N.Y.: Springer-Verlag; 1997.
Lieberman J A, Sheitman B B, Kinon B J. Neurochemical sensitization in the pathophysiology of schizophrenia: deficits and dysfunction in neuronal regulation and plasticity. Neuropsychopharmacology. 1997 October; 17(4):205-29.
Lin G McKay G, Hubbard J W, Midha K K. Decomposition of clozapine N-oxide in the qualitative and quantitative analysis of clozapine and its metabolites. J. Pharm. Sci. 83: 1412-7 (1994).
Marls, R W: The relation of no-fatal suicide attempts to completed suicides, in Assessment and Predication of Suicide, Edited by Maris R W, Berman A L Maltensberger J T Yuft R L New York, Guilford Press 1992, pp 362-80.
McCully C L, Balis F M, Bacher J, Phillips J, Poplack D G. A rhesus monkey model for continuous infusion of drugs into cerebrospinal fluid. Lab Anim Sci. 1990 September; 40(5):520-5.
Mechiel Korte S. De Boer S F. A robust animal model of state anxiety: fear-potentiated behaviour in the elevated plus-maze. European Journal of Pharmacology. 463(1-3):163-75, 2003
M J McLeish, B Capuano, and E J Lloyd “Clozapine” in Analytical Profiles of Drug Substances and Excipients p 145-184. Academic Press, 1993.
Miner L L, Marks M J, Collins A C. Genetic analysis of nicotine-induced seizures and hippocampal nicotinic receptors in the mouse. J Pharmacol. Exp. Ther., 239:853-860, 1986.
Nagamoto H T, Adler L E, McRae K A, Heuttl P, Cawthra E, Gerhardt G, Hea R, Griffith J. Auditory P50 in schizohrenics on clozapine: improved gating parallels clinical improvement and changes plasma 3-methoxy-4-hydroxyphenylglycol. Neuropsychobiol, 39:10-17, 1999.
Narrow W E. One-year prevalence of mental disorders, excluding substance use disorders, in the U.S.: NIMH ECA prospective data. Population estimates based on U.S. Census estimated residential population age 18 and over on Jul. 1, 1998. Unpublished.
Nordin C, Alme B, Bondesson U. CSF and serum concentrations of clozapine and its demethyl metabolite: a pilot study. Psychopharmacology (Berl). 1995 November; 122(2): 104-7.
Olincy A, H a s J G, Johnson L L, Pender V, Kongs S, Allensworth D, Ellis J, Zerbe G O, Leonard S, Stevens Kem Stevens J O, martin L, Alder L E, Soti F, Kem W R, Freedman R. Proof-of-principle trial of an α 7 cholinergic agonist for neurocognitive dysfunction in schizophrenia. Nature Med, 2005 (in review)
O'Neill H C, Reiger K, Kem W. R, Stevens, K E. DMXB, an a7 nicotinic agonist, normalizes auditory gating in isolation reared rats. Psychopharmacol, 169:332-339, 2003
Paice J A, et al: Intraspinal morphine for chronic pain: a retrospective, multicenter study. J Pain Symptom Manage. 1996 February; 11(2):71-80.
Pickering L K, Ericsson C D, Ruiz-Palacios G, Blevins J, Miner M E. Intraventricular and parenteral gentamicin therapy for ventriculitis in children. Am J Dis Child. 1978 May; 132(5):480-3.
Poplack D G, Bleyer W A, Wood J H, Kostolich M, Savitch J L, Ommaya A K A primate model for study of methotrexate pharmacokinetics in the central nervous system. Cancer Res. 1977 July; 37(7 Pt 1):1982-5.
Reid William H., M. D., M. P. H., Mark Mason, M. S, and Thomas Hogan, M. B. A. Suicide Prevention Effects Associated With Clozapine Therapy in Schizophrenia and Schizoaffective Disorder Psychiatr Serv 49:1029-1033, August 1998.
Rupp A, Keith S J. The costs of schizophrenia. Assessing the burden. Psychiatr Clin North Am. 1993 June; 16(2):413-23.
Russig H. Pezze M A. Nanz-Bahr N I. Pryce C R. Feldon J. Murphy C A. Amphetamine withdrawal does not produce a depressive-like state in rats as measured by three behavioral tests. Behavioural Pharmacology. 14(1):1-18, 2003

Simosky, J. K., Stevens, K. E., Adler, L. E. and Freedman, R. Clozapine improves deficient inhibitory auditory processing in DBAJ2 mice, via a nicotinic cholinergic mechanism. Psychopharmacol, 165: 386-396, 2002.

Simosky J K, Stevens K B, Kem, W R and Freedman R. Intragastric DMXB-A, an □7 nicotinic agonist, improves deficient sensory inhibition in DBAI2 mice. Biol. Psychiat. 50:493-500, 2001.

Stein L I, Test M A. Alternative to mental hospital treatment. I. Conceptual model, treatment program, and clinical evaluation. Arch Gen Psychiatry. 1980.

Stevens K E, Freedman R, Collins A C, Hall M, Leonard S, Marks M J, and Rose G M. Genetic correlation of hippocampal auditory evoked response and a-bungarotoxin binding in inbred mouse strains. Neuropsychopharmacol., 15: 152-162, 1996.
Stevens K E, Kem W R, Freedman R. Selective a7 nicotinic receptor stimulation normalizes chronic cocaine-induced loss of hippocampal sensory inhibition in C3H mice. Biol. Psychiat., 46:1443-1450, 1999.
Stevens K E, Kem W R, Mahnir V and Freedman R. Selective a7 nicotinic agonists normalize inhibition of auditory response in DBA mice, Psychopharmacol. 136:320-327, 1998.
Stitzel J A, Farnham D A, Collins A C. Linkage of strain-specific nicotinic receptor subunit restriction fragment length polymorphisms with levels of a-bungarotoxin binding in brain. Mol Brain Res 43:30-40, 1996.
Stitzel J A, Jenkins P M, Lautner M A: CHRNA7 promotor polymorphisms in mice affect gene expression in a cell-type specific fashion. Program No. 465.8. 2003 Abstract Viewer/Itinerary Planner. Washington, D.C.: Soc Neurosci, 2003.
Test M A, Stein L I. Alternative to mental hospital treatment. III. Social cost. Arch Gen Psychiatry. 1980 April; 37(4):409-12.
Urca G, Frenk H. Intracerebral opiates block the epileptic effect of intracerebroventricular (ICV) leucine-enkephalin. Brain Res. 1983 Jan. 17; 259(1):103-10.
Venables P. Input dysfunction in schizophrenia. In: B. A. Maher, ed. Progress in Experimental Personality Research, Orlando: Academic Press; 1964: 1-47.
Venables P H. Hippocampal function and schizophrenia. Experimental psychological evidence. Ann. N.Y. Acad. Sci., 658: 111-127, 1992.
Vera P L, Miranda-Sousa A J, Ordorica R C, Nadelhaft I. Central effects of clozapine in regulating micturition in anesthetized rats. BMC Pharmacol. 2002; 2:6. Epub 2002 Mar. 7.
Volavka J, Czobor P, Sheitman B, Lindenmayer J P, Citrome L, McEvoy J P, Cooper T B, Chakos M, Lieberman J A. Clozapine, olanzapine, risperidone, and haloperidol in the treatment of patients with chronic schizophrenia and schizoaffective disorder. Am J Psychiatry. 2002 February; 159(2):255-62. Erratum in: Am J Psychiatry 2002 December; 159(12):2132.
Waterworth D M, Bassett A S, Brzustowicz L M. Recent advances in the genetics of schizophrenia. Cell Mol Life Sci, 59:331-348, 2002.
Weisbrod B A, Test M A, Stein L I. Alternative to mental hospital treatment. II. Economic benefit-cost analysis. Arch Gen Psychiatry. 1980 April; 37(4):400-5.
Wood J H, Poplack D G, Bleyer W A, Ommaya A K. Primate model for long-term study of intraventricularly or intrathecally administered drugs and intracranial pressure. Science. 1977 Feb. 4; 195(4277):499-501.
Yaksh T L. Spinal systems and pain processing: development of novel analgesic drugs with mechanistically defined models. Trends Pharmacol Sci 1999; 20:329-37.
Yaksh T L, Horais K A, Tozier N A, Allen J W, Rathbun M, Rossi S S, Sommer C, Meschter C, Richter P J, Hildebrand K R. Chronically infused intrathecal morphine in dogs. Anesthesiology. 2003 July; 99(1):174-87.
Yaksh T L, Allen J W. The use of intrathecal midazolam in humans: a case study of process. Anesth Analg. 2004 June; 98(6):1536-45.
Yaksh T L, Allen J W. Preclinical insights into the implementation of intrathecal midazolam: a cautionary tale. Anesth Analg. 2004 June; 98(6):1509-11.
Walker E, Kestler L, Bollini A, Hochman K M. Schizophrenia: etiology and course. Annu Rev Psychol. 2004; 55:401-30.

Wu E Q, Birnbaum H G, Shi L, Ball D E, Kessler R C, Moulis M, Aggarwal J. The Economic Burden of Schizophrenia in the United States in 2002, J Clin Psychiatry. 2005 September; 66(9): 1122-1129.

What is claimed:

1. A method for treating a CNS-related condition or disorder in a subject in need thereof, the method comprising intracerebroventricularly administering to the subject a pharmaceutical composition comprising (i) a CNS therapeutic agent effective to treat the CNS-related condition or disorder and (ii) a solubility enhancing agent; wherein the solubility enhancing agent allows an effective amount of the CNS therapeutic agent to be intracerebroventricularly administered to the subject; and wherein the CNS therapeutic agent maintains solubility in the composition for at least two months at physiological temperature and pH.

2. The method of claim 1, wherein the CNS-related condition or disorder is selected from the group consisting of epilepsy, schizophrenia, closed head injury spectrum, Alzheimer's disease spectrum, sleep disorders spectrum, depression, anxiety spectrum, bipolar disorder, and multiple sclerosis.

3. The method of claim 1, wherein the pharmaceutical composition is chronically administered over at least two months via an implantable delivery device.

4. The method of claim 1, wherein the subject is selected from the population of individuals who are refractory to treatment via systemic administration of the CNS therapeutic agent.

5. The method of claim 1, wherein the refractory subject shows an alleviation of one or more symptoms when treated by intracerebroventricular administration of the pharmaceutical composition.

6. The method of claim 1, wherein the subject is administered a dosage of the CNS therapeutic agent significantly reduced, as compared to the dosage required when administered systemically.

7. The method of claim 1, wherein the dosage of CNS therapeutic agent is at an intracerebroventricular administration to systemic administration ratio of about 1:250 to about 1:600.

8. The method of claim 1, wherein the CNS therapeutic agent maintains solubility in cerebral spinal fluid upon administration to the subject.

9. The method of claim 1, wherein the CNS therapeutic agent is active in the treatment of epilepsy.

10. The method of claim 9, wherein the CNS therapeutic agent is an anti-epilepsy agent that acts on the GABA system, a sodium channel, and/or a calcium channel.

11. The method of claim 9, wherein the CNS therapeutic agent is selected from the group consisting of felbamate, lamictal, bumex, tegretol, valproate, adenosine, pharmaceutically acceptable salts, esters, and acids thereof, and combinations thereof.

12. The method of claim 1, wherein the CNS therapeutic agent is active in the treatment of schizophrenia.

13. The method of claim 12, wherein the CNS therapeutic agent is an anti-schizophrenic agent that acts as a nicotinic direct or indirect agonist, or a dopamine antagonist.

14. The method of claim 12, wherein the CNS therapeutic agent is selected from the group consisting of clozapine, ondansetron, olanzapine, risperidone, pharmaceutically acceptable salts, esters, and acids thereof, and combinations thereof.

15. The method of claim 1, wherein the CNS therapeutic agent is active in the treatment of depression and/or anxiety.

16. The method of claim 15, wherein the CNS therapeutic agent is an anti-depression and/or anti-anxiety agent that affects adrenergic and serotonergic activity.

17. The method of claim 15, wherein the CNS therapeutic agent is selected from the group consisting of phenelzine, fluoxetine, tranylcypromine, amitryptyline, clomipramine, isocarboxazid, pharmaceutically acceptable salts, esters, and acids thereof, and combinations thereof.

18. The method of claim 1, wherein the solubility enhancing agent is selected from the group consisting of cyclodextrin, octylglucoside, Tween 20, sucrose ester, pluronic F-68, and combinations thereof.

19. The method of claim 18, wherein the cyclodextrin is β-hydroxypropyl-cyclodextrin.

20. The method of claim 1, wherein the subject is a human, and wherein the intracerebroventricular administered dose to the human is about 30-fold lower to about 30-fold higher than the effective ICV dose in rats.

21. The method of claim 20, wherein the CNS therapeutic agent is felbamate and the effective ICV dose in rats is about 0.08242 mg/kg.

22. The method of claim 20, wherein the CNS therapeutic agent is clozapine and the effective ICV dose in rats is about 0.040 mg/kg.

23. The method of claim 1, wherein the CNS therapeutic agent is a hydrophobic compound.

24. An apparatus comprising (i) an intracerebroventricular delivery device, (ii) a central nervous system (CNS) therapeutic agent, and (iii) a solubility enhancing agent; wherein the solubility enhancing agent allows an effective amount of the CNS therapeutic agent to be intracerebroventricularly administered to a subject; and wherein the CNS therapeutic agent maintains solubility in the presence of the solubility enhancing agent for at least two months at physiological temperature and pH.

25. The apparatus of claim 24, wherein the CNS therapeutic agent is active in the treatment of a CNS condition or disorder selected from the group consisting of epilepsy, schizophrenia, closed head injury spectrum, Alzheimer's disease spectrum, sleep disorders spectrum, depression, anxiety spectrum, bipolar disorder, and multiple sclerosis.

26. The apparatus of claim 24, wherein the solubility enhancing agent is selected from the group consisting of cyclodextrin, octylglucoside, Tween 20, sucrose ester, pluronic F-68, and combinations thereof.

27. The apparatus of claim 24, wherein the solubility enhancing agent is present in an amount ranging from about 2% to about 25% by weight.

28. The apparatus of claim 24, wherein the CNS therapeutic agent to solubility enhancing agent molar ratio is between about 1:1 and about 1:10.

29. The apparatus of claim 24, wherein the CNS therapeutic agent is present at a concentration greater than corresponding concentrations suitable for systemic administration.

30. The apparatus of claim 24, further comprising an antioxidant.

31. The apparatus of claim 24, wherein the CNS therapeutic agent maintains CNS therapeutic agent stability in cerebral spinal fluid upon intracerebroventricular administration to a subject.

32. The apparatus of claim 24, wherein the CNS therapeutic agent maintains solubility in cerebral spinal fluid upon intracerebroventricular administration to a subject.

33. The apparatus of claim 24, wherein the delivery device is an implantable pump.

34. The apparatus of claim 24, wherein the subject is a human, and wherein the intracerebroventricular administered dose to the human is about 30-fold lower to about 30-fold higher than the effective ICV dose in rats.

35. The apparatus of claim 24, wherein the CNS therapeutic agent is a hydrophobic compound.

36. A kit comprising (i) an intracerebroventricular delivery device, (ii) an amount of a central nervous system (CNS) therapeutic agent suitable for intracerebroventricular administration to a subject in need thereof, (iii) a solubility enhancing agent, and (iv) instructions for using the kit to treat a CNS-related condition or disorder in the subject; wherein the solubility enhancing agent allows an effective amount of the CNS therapeutic agent to be intracerebroventricularly administered to the subject; and wherein the CNS therapeutic agent maintains solubility in the presence of the solubility enhancing agent for at least two months at physiological temperature and pH.

37. The kit of claim 36, wherein the CNS therapeutic agent is active in the treatment of epilepsy.

38. The kit of claim 37, wherein the CNS therapeutic agent is felbamate.

39. The kit of claim 36, wherein the CNS therapeutic agent is active in the treatment of schizophrenia.

40. The kit of claim 39, wherein the CNS therapeutic agent is clozapine.

41. The kit of claim 36, wherein the solubility enhancing agent is selected from the group consisting of cyclodextrin, octylglucoside, Tween 20, sucrose ester, pluronic F-68, and combinations thereof.

42. The kit of claim 41, wherein the cyclodextrin is β-hydroxypropyl-cyclodextrin.

43. The kit of claim 36, wherein the CNS therapeutic agent to solubility enhancing agent molar ratio is between about 1:1 and about 1:10.

44. The kit of claim 36, wherein the CNS therapeutic agent is present at a concentration greater than corresponding concentrations suitable for systemic administration.

45. The kit of claim 36, further comprising an antioxidant.

46. The kit of claim 36, wherein the CNS therapeutic agent maintains CNS therapeutic agent stability in cerebral spinal fluid upon intracerebroventricular administration to the subject.

47. The kit of claim 36, wherein the CNS therapeutic agent maintains solubility in cerebral spinal fluid upon intracerebroventricular administration to a subject.

48. The kit of claim 36, wherein the delivery device is an implantable pump.

49. The kit of claim 36, wherein the subject is a human, and wherein the intracerebroventricular administered dose to the human is about 30-fold lower to about 30-fold higher than the effective ICV dose in rats.

50. The kit of claim 49, wherein the CNS therapeutic agent is felbamate and the effective ICV dose in rats is about 0.08242 mg/kg.

51. The kit of claim 49, wherein the CNS therapeutic agent is clozapine and the effective ICV dose in rats is about 0.040 mg/kg.

52. The kit of claim 36, wherein the CNS therapeutic agent is a hydrophobic compound.

53. The kit of claim 36, further comprising a penetration enhancing excipient.
